(19) United States
(12) Patent Application Publication
(10) Pub. No.: US 2024/0376363 A1
(43) Pub. Date: Nov. 14, 2024

(54) FLUORINE SUBSTITUTED CYCLOBUTENE COMPOUNDS, AND COMPOSITIONS, METHODS AND USES INCLUDING SAME

(71) Applicant: HONEYWELL INTERNATIONAL INC., Charlotte, NC (US)

(72) Inventors: Yian Zhai, Charlotte, NC (US); Ryan Hulse, Charlotte, NC (US); Rajiv Ratna Singh, Charlotte, NC (US); David Nalewajek, Charlotte, NC (US)

(73) Assignee: HONEYWELL INTERNATIONAL INC., Chalotte, NC (US)

(21) Appl. No.: 18/696,291

(22) PCT Filed: Sep. 27, 2022
(86) PCT No.: PCT/US2022/044904

§ 371 Date: Mar. 27, 2024

Related U.S. Application Data
(60) Provisional application No. 63/248,990, filed on Sep. 27, 2021.

(51) Int. Cl. C09K 5/04; C07C 23/06; C07C 43/192; C07C 43/247; F25B 9/00; H01M 10/613; H01M 10/625; H01M 10/6569; H01M 10/663

(52) U.S. Cl. CPC C09K 5/044; C07C 23/06; C07C 43/192; C07C 43/247; H01M 10/613; H01M 10/625; H01M 10/6569; H01M 10/663; C07C 2601/04; F25B 9/002

(57) ABSTRACT

A heat transfer composition for transferring heat and/or energy to and/or from an article, device or fluid, wherein the heat transfer composition comprises, consists essentially of or consists of a compound according to Formula I or Formula II or Formula III:

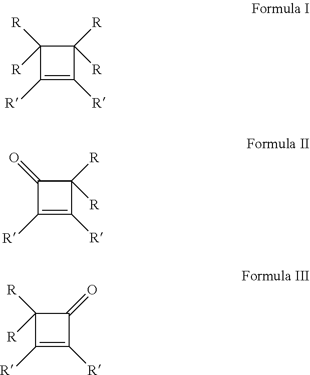

where each R and each R′ is independently selected from H, F, Cl, R1, and OR1, each R1 is independently: a C1 to C3 alkane that is unsubstituted or substituted with F and/or Cl; or C1 to C3 alkene that is unsubstituted or substituted with F and/or Cl; or Bz, where Bz is an unsubstituted benzene ring, and provided that the molecule has at least three F substituents bonded to a carbon in the cyclobutene ring.

### CROSS-REFERENCE TO RELATED APPLICATIONS

This application claims priority to U.S. Provisional Application No. 63/084,280 filed Sep. 28, 2020, the entire contents of which are incorporated herein by reference.

### FIELD

The present invention relates to fluorine substituted cyclobutene compounds, including novel fluorine substituted cyclobutene compounds, to compositions containing same, and to methods and uses of such compounds and compositions in heat transfer applications, including: temperature control in manufacture of electronic equipment; thermal management of operating electronic devices and power systems, and avionic and military cooling.

### BACKGROUND

Certain halogen substituted cyclobutene compounds are known to exist. For example, U.S. 5,233,105 disclosed that 1,2-dichloro-3,3,4,4-tetrafluorocyclobut-1-ene is formed as a reaction product in the catalytic reaction of hexachlorobutadiene, but no use is disclosed for this compound. U.S. Pat. No. 5,041,304 discloses methods for treatment of sheets, fibers and the like to impart water repellancy with a treating gas that may include 1,2-dichlorotetrafluorocyclobutene and hexafluorocyclobutene. U.S. Pat. No. 7,179,413 discloses a process for forming synthetic fiber polymers from flash spinning using a co-spinning agent that might include 1H, 2H-perfluorocyclobutene.

Applicants have come to appreciate that many challenging issues are associated with the development of new compounds and compositions for use in many important applications. For example, in portable and hand-held electronic devices, the desire to miniaturize while adding functionality increases the thermal power density of the device while in operation, thus making cooling of the electronics components within such devices, including the batteries, more challenging. As general rule, increases in computational power within desktop computers, data centers, telecommunications centers and the like results in an increase in the heat output when such devices are operating, again making thermal management of such electronic devices increasingly important and increasingly more difficult and demanding. Other examples of thermal management challenges occur as a result of the increasing use of electronic vehicles, including particularly, cars, trucks, motorcycles and the like. In electronic vehicles the thermal management function is especially important and challenging for several reasons, including the criticality of cooling and/or heating the batteries to be within a relatively narrow temperature range and in a way that is reliable, efficient and safe, and the challenge to provide effective thermal battery management is becoming greater as the demand for battery-operated vehicles with greater range and faster charging increases.

The efficiency and effectiveness of batteries, especially the batteries that provide the power in electronic vehicles, is a function of the operating temperature at which they operate. Thus, thermal management system must frequently be able to do more than simply remove heat from the battery during operation and/or charging—it must be able to effect cooling in a relatively narrow temperature range using equipment that is as low cost as possible and as light weight as possible. This results in the need for a heat transfer fluid in such systems that possesses a difficult-to-achieve combination of physical and performance properties. Furthermore, in some important applications the thermal management system must be able to add heat to the battery, especially as the vehicle is started in cold weather, which adds further to the difficulty of discovering and developing/obtaining compounds and/or compositions effective in such systems, not only from a thermal performance standpoint, but also a myriad of other standpoints, including environmental, safety (flammability and toxicity), dielectric properties, and others.

As a particular example of the importance of dielectric constant, one frequently used system for the thermal management of electronic vehicle batteries involves immersing the battery in the fluid used for thermal management. Such systems add the additional constraint that the fluid used in such systems must be electronically compatible with the intimate contact with the battery, or other electronic device or component, while the battery or device is in operation. In general, this means the fluid must not only be non-flammable, it must also have a low electrical conductivity and a high level of stability while in contact with the battery or other electronic component while the component is operating and at the relatively high temperatures existing during operation. Applicants have come to appreciate the desirability of such properties even in indirect cooling of operating electronic devices and batteries because leakage of any such fluid may result in contact with operating electronic components.

Perfluorinated compounds have heretofore frequently been used in many of these demanding applications. For example, the thermal management fluid which has been commonly used for battery cooling, including immersive cooling, is a water/glycol combination, although other classes of materials, including chlorofluorocarbons, fluorohydrocarbons, chlorohydrocarbons and hydrofluoroethers, have been mentioned for possible use. See, for example, US 2018/0191038.

Vapor phase soldering is another example of a process that utilizes heat transfer fluids. In this application, high temperatures are used and accordingly the heat transfer fluid must be suitable for high temperature exposure (e.g., up to 250° C.) Currently, perfluoropolyethers (PFPE) are commonly used as the heat transfer fluids in this application. Although many PFPEs have adequate thermal stability for these high temperatures, they are environmentally persistent with extremely long atmospheric lifetimes which, in turn, gives rise to high global warming potentials (GWPs).

Thus, applicants have come to appreciate the need for thermal management methods and systems which use a heat transfer fluid which is environmentally acceptable (low GWP and low ODP), non-flammable, has low or no toxicity, has excellent insulating properties and has thermal properties that provide effective cooling and/or heat, including at relatively high temperatures and/or for use in operating electronic components in a relatively narrow temperature range with equipment that preferably low cost, reliable and light weight, among other uses. Thus, for example, applicants have found that fluids that have relatively low boiling points (e.g., below 50° C.) are not desirable in many applications since the use of such fluids will tend to increase the cost and/or weight of the cooling equipment for many battery and/or electronic cooling applications, and may also decrease reliability, as explained hereinafter.

The Rankine cycle is the standard thermodynamic cycle in general use for electric power generation. The essential elements of a Rankine cycle system are: 1) a boiler to change liquid to vapor at high pressure; 2) a turbine to expand the vapor to derive mechanical energy; 3) a condenser to change low pressure exhaust vapor from the turbine to low pressure liquid; and 4) a pump to move condensate liquid back to the boiler at high pressure.

Various working fluids have been suggested for use in Rankine cycles, including HFC-245fa. However, there is a desire in the industry to provide a working fluid which is environmentally acceptable, has excellent thermodynamic properties, and can operate efficiently over a wide range of heat source temperatures, including, for example, at least about 200° C., for example of from about 200° C. to about 400° C.

There is also a desire in the industry to provide a heat transfer fluid (e.g., a refrigerant) which is environmentally acceptable, has excellent thermodynamic properties, and is non-flammable.

### SUMMARY

The present invention includes heat transfer compositions for transferring heat and/or energy to and/or from an article, device or fluid, wherein said heat transfer fluid comprises, consists essentially of or consists of one or more Group A Compounds. As the term is used herein, a Group A Compound is a compound according to Formula I or Formula II or Formula III:

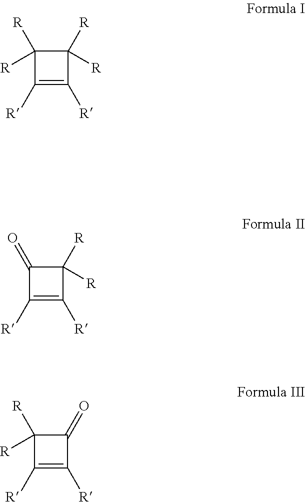

where each R and each R′ is independently selected from H, F, Cl, R1, and OR1, each R1 is independently: a C1 to C3 alkane that is unsubstituted or substituted with F and/or Cl; or C1 to C3 alkene that is unsubstituted or or substituted with F and/or Cl; or Bz, where Bz is an unsubstituted benzene ring, and provided that the molecule has at least three F substituents bonded to a carbon in the cyclobutene ring. In case cis and trans stereo isomers exist, either or both isomers may be used in this application. For the purposes of convenience, compositions according to this paragraph are sometimes referred to herein as Composition 1.

For the purposes of convenience, the abbreviation “HTC” is sometimes used herein to refer to “heat transfer composition,” and unless specifically indicated hereinafter, or unless the context requires otherwise, the term HTC means a heat transfer fluid for transferring heat and/or energy to and/or from an article, device or fluid.

The present invention includes HTCs having a boiling point of from about 25° C. to about 150° C. and which comprise, consist essentially of or consist of one or more Group A Compounds. For the purposes of convenience, HTCs according to this paragraph are sometimes referred to herein as Composition 2.

The present invention includes HTCs having a boiling point of from about 25° C. to about 150° C. and a dielectric constant of less than about 8, and which comprise, consist essentially of or consist of one or more Group A Compounds. For the purposes of convenience, compositions according to this paragraph are sometimes referred to herein as Composition 3A.

As used herein, the term “dielectric strength” refers to the breakdown voltage in kV as measured in accordance with ASTM D87-13, Procedure A, with the modification that the spacing between the electrodes is 2.54 mm and the rate of rise was 500 V/sec.

The present invention includes HTCs having a boiling point of from about 25° C. to about 150° C. and a dielectric constant of less than about 5 and which comprise, consist essentially of or consist of one or more Group A Compounds. For the purposes of convenience, compositions according to this paragraph are sometimes referred to herein as Composition 3B.

The present invention includes HTCs having a boiling point of from about 25° C. to about 150° C. and a dielectric constant of less than about 2.5 and which comprise, consist essentially of or consist of one or more Group A Compounds. For the purposes of convenience, compositions according to this paragraph are sometimes referred to herein as Composition 3C.

The present invention includes HTCs having a boiling point of from about 25° C. to about 150° C., a Global Warming Potential (GWP) of less than about 150 and a dielectric constant of less than about 2.5 and which comprise, consist essentially of or consist of one or more Group A Compounds. For the purposes of convenience, compositions according to this paragraph are sometimes referred to herein as Composition 3D.

The present invention includes HTCs having a boiling point of from about 25° C. to about 150° C., a Global Warming Potential (GWP) of less than about 75 and a dielectric constant of less than about 2.5 and which comprise, consist essentially of or consist of one or more Group A Compounds. For the purposes of convenience, compositions according to this paragraph are sometimes referred to herein as Composition 3E.

The present invention includes HTCs having a boiling point of from about 25° C. to about 150° C. and a dielectric strength of at least about 30 and which comprise, consist essentially of or consist of one or more Group A Compounds. For the purposes of convenience, compositions according to this paragraph are sometimes referred to herein as Composition 4A.

The present invention includes HTCs having a boiling point of from about 25° C. to about 150° C. and a dielectric strength of at least about 40 and which comprise, consist essentially of or consist of one or more Group A Compounds. For the purposes of convenience, compositions according to this paragraph are sometimes referred to herein as Composition 4B.

The present invention includes HTCs having a boiling point of from about 25° C. to about 150° C., a dielectric strength of at least about 30, and a thermal conductivity of at least about 0.055 W/m−K, and which comprise, consist essentially of or consist of one or more Group A Compounds. For the purposes of convenience, compositions according to this paragraph are sometimes referred to herein as Composition 4C.

As used herein, the term “thermal conductivity” refers to the breakdown voltage in kV as measured in accordance with ASTM D7896-19 .

The present invention includes HTCs having a boiling point of from about 25° C. to about 150° C., a dielectric strength of at least about 40, and a thermal conductivity of at least about 0.065 W/m−K, and which comprise, consist essentially of or consist of one or more Group A Compounds. For the purposes of convenience, compositions according to this paragraph are sometimes referred to herein as Composition 4D.

The present invention includes HTCs having a boiling point of from about 25° C. to about 150° C., a Global Warming Potential (GWP) of less than about 150, a dielectric strength of at least about 40, and a thermal conductivity of at least about 0.065 W/m−K and which comprise, consist essentially of or consist of one or more Group A Compounds. For the purposes of convenience, compositions according to this paragraph are sometimes referred to herein as Composition 4E.

The present invention includes HTCs having a boiling point of from about 25° C. to about 150° C., a Global Warming Potential (GWP) of less than about 75, a dielectric strength of at least about 40, and a thermal conductivity of at least about 0.065 W/m−K and which comprise, consist essentially of or consist of one or more Group A Compounds. For the purposes of convenience, compositions according to this paragraph are sometimes referred to herein as Composition 4F.

The present invention includes methods for removing heat and/or energy from an article, device or fluid or adding heat and/or energy to an article, device or fluid comprising:
(a) providing the article, device or fluid in heat transfer contact with the source of heat and/or energy; and
(b) transferring said heat and/or energy from any composition according to any of Compositions 1-4. For the purposes of convenience, methods according to this paragraph are sometimes referred to herein as Heat Transfer Methods 1.

As used herein, reference to a group of compositions, methods, and the like, defined by numbers, such as the reference in the preceding paragraph to “any of Compositions 1-4” specifically includes all such numbered compositions, including all numbered compositions with a suffix. For example, the reference to “any of Compositions 1-4” includes each of Composition 1, Composition 2, Composition 3A, Composition 3B, Composition 3C, Composition 3D, Composition 3E, Composition 4A, Composition 4B, Composition 4C, Composition 4D, Composition 4E and Composition 4F.

The present invention includes systems and devices that include a heat transfer fluid for transferring heat within and/or to and/or from that system or device, said system and/or device comprising as the heat transfer fluid a composition according to any of Compositions 1-4. For the purposes of convenience, systems and/or devices according to this paragraph are sometimes referred to herein as System 1.

The present invention includes HTCs that comprise, consist essentially of or consist of one or more Group A1 Compounds. As the term is used herein, a Group A1 Compound is a compound selected from among Compound 1 through Compound 18 as defined in the following Table A1:

TABLE A1

| | COMPOUND | |
|---|---|---|
| STRUCTURE* | NUMBER | CAS No.** |
| 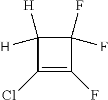 1-chloro-2,3,3-trifluorocyclo- 1-ene | 1 | 694-62-2 |
| 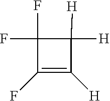 1,4,4-trifluorocyclobut- 1-ene | 2 | 3932-66-9 |
| 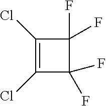 1,2-dichloro-3,3,4,4- tetrafluorocyclobut-1-ene | 3 | 377-93-5 |
| 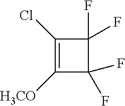 [SMILES: COC1=C(Cl)C(F)(F)C1(F)F] 1-chloro-3,3,4,4- tetrafluoro-2- methylcyclobut-1-ene | 4 | 359-97-7 |
| 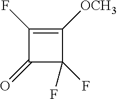 2,4,4-trifluoro-3- methoxycyclobut-2-en-1-one | 5 | 60407-10-5 |
| 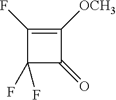 3,4,4-trifluoro-2- methoxycyclobut-2-en-1-one | 6 | 60376-36-5 |
| 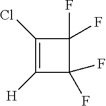 1-chloro-3,3,4,4- tetrafluorocyclobut-1-ene | 7 | 695-44-3 |
| 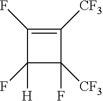 [SMILES: FC1=C(C(F)(F)F)C(F)(C(F)(F)F)C1F] cis-1,3,4-trifluoro-2,3- bis(trifluoromethyl) cyclobut-1-ene | 8A | New compound |
| 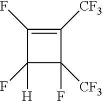 [SMILES: FC1=C(C(F)(F)F)C(F)(C(F)(F)F)C1F] trans-1,3,4-trifluoro-2,3- bis(trifluoromethyl) cyclobut-1-ene | 8B | New compound |
| 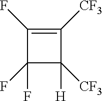 [SMILES: FC1=C(C(F)(F)F)C(C(F)(F)F)C1(F)F] 2,3,3-trifluoro-1,4- bis(trifluoromethyl) cyclobut-1-ene | 9 | New compound |
| 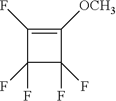 [SMILES: COC1=C(F)C(F)(F)C1(F)F] 1,3,3,4,4-pentafluoro- 2-methoxycyclobut- 1-ene | 10 | 359-98-8 |
| 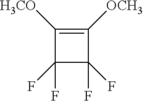 [SMILES: COC1=C(OC)C(F)(F)C1(F)F] 3,3,4,4-tetrafluoro-1,2- dimethoxycyclobut-1-ene | 11 | 361-82-0 |
| 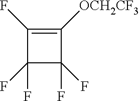 1,3,3,4,4-pentafluoro-2- (trifluoromethoxy)cyclobut- 1-ene | 12 | 25353-05-3 |
| 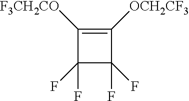 3,3,4,4-pentafluoro-1,2- bis(trifluoromethoxy)cyclobut- 1-ene | 13 | 25353-07-5 |
| 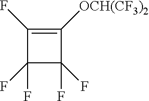 1,3,3,4,4-pentafluoro-2- ((1,1,1,3,3,3-hexafluoropropan- 2-yl)oxy)cyclobut-1-ene | 14 | 25353-06-4 |
| 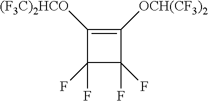 3,3,4,4-pentafluoro- 1,2-bis((1,1,1,3,3,3- hexafluoropropan-2-yl)oxy) cyclobut-1-ene | 15 | New compound |
| 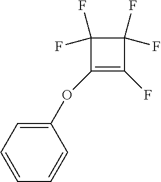 [SMILES: FC1=C(Oc2ccccc2)C(F)(F)C1(F)F] ((perfluorocyclobut- 1-en-1-yl)oxy)benzene | 16 | NA |
| 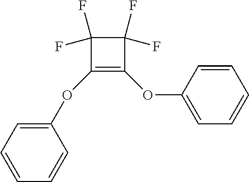 [SMILES: FC1(F)C(Oc2ccccc2)=C(Oc2ccccc2)C1(F)F] ((perfluorocyclobut-1-ene-1,2-diyl) bis(oxy))dibenzene | 17 | New compound |
| 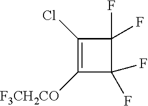 1-chloro-3,3,4,4-tetrafluoro-2- (2,2,2-trifluoroethoxy) cyclobut-1-ene | 18 | New compound |

*The depicted structure is intended to cover all stereoisomers of the depicted
compound, and compound name is provided for convenience and in the event
of an inconsistency the depicted structure controls.
**The present invention includes novel compounds, as described in detail below,
which do not have CAS numbers.

For the purposes of convenience, compositions according to this paragraph are sometimes referred to herein as Composition 5A.

The present invention also includes HTCs that comprise, consist essentially of or consist of one or more Group A2 Compounds. As the term is used herein, a Group A2 Compound is a compound selected from among Compound 2 and Compounds 4-18 as defined in Table A1 above. Applicants have found that this group of compounds is especially preferred for applications in which low toxicity is highly desirable since Compound 1 has been tested and failed long term (28 day) toxicity exposure and that Compound 3 has been tested and failed short term (4 hour) toxicity exposure. For the purposes of convenience, compositions according to this paragraph are sometimes referred to herein as Composition 5B.

The present invention also includes HTCs that comprise, consist essentially of or consist of one or more a Group A Compound, provided however, that the composition is essentially free of Compound 1 and of Compound 3. For the purposes of convenience, compositions according to this paragraph are sometimes referred to herein as Composition 5C.

The present invention also includes HTCs that comprise, consist essentially of or consist of one or more a Group A Compound, provided however, that no R′ is Cl. For the purposes of convenience, compositions according to this paragraph are sometimes referred to herein as Composition 5D.

The present invention also includes HTCs that comprise, consist essentially of or consist of one or more a Group A Compound, provided however, that the composition is essentially free of any Group A Compound in which R′ is Cl. For the purposes of convenience, compositions according to this paragraph are sometimes referred to herein as Composition 5E. The present invention includes HTCs having a boiling point of from about 25° C. to about 150° C. and which comprise, consist essentially of or consist of one or more Group A1 Compounds. For the purposes of convenience, compositions according to this paragraph are sometimes referred to herein as Composition 6.

The present invention includes HTCs having a boiling point of from about 25° C. to about 150° C. and a dielectric constant of less than about 8, and which comprise, consist essentially of or consist of one or more Group A1 Compounds. For the purposes of convenience, compositions according to this paragraph are sometimes referred to herein as Composition 7.

The present invention includes HTCs having a boiling point of from about 25° C. to about 150° C. and a dielectric strength of at least about 40 and which comprise, consist essentially of or consist of one or more Group A1 Compounds. For the purposes of convenience, compositions according to this paragraph are sometimes referred to herein as Composition 8.

The present invention includes methods for removing heat and/or energy from an article, device or fluid or adding heat and/or energy to an article, device or fluid comprising:
(a) providing the article, device or fluid in heat transfer contact with the source of heat and/or energy; and
(b) transferring said heat and/or energy from any composition according to any Compositions 5-8. For the purposes of convenience, methods according to this paragraph are sometimes referred to herein as Heat Transfer Methods 2.

The present invention includes systems and devices that include a heat transfer fluid for transferring heat within and/or to and/or from that system or device, said system and/or device comprising as the heat transfer fluid a composition according to any of Compositions 5-8. For the purposes of convenience, methods according to this paragraph are sometimes referred to herein as System 2.

The present invention includes HTCs that comprise, consist essentially of or consist of one or more Group A2 Compounds. As the term is used herein, a Group A2 Compound is a compound selected from Compound 1, Compound 3, Compound 7, Compound 10, Compound 12 and Compound 14. For the purposes of convenience, compositions according to this paragraph are sometimes referred to herein as Composition 9.

The present invention includes HTCs having a boiling point of from about 50° C. to about 100° C. and which comprise, consist essentially of or consist of one or more Group A2 Compounds. For the purposes of convenience, compositions according to this paragraph are sometimes referred to herein as Composition 10.

The present invention includes HTCs having a boiling point of from about 50° C. to about 100° C. and a dielectric constant of less than about 8, and which comprise, consist essentially of or consist of one or more Group A2 Compounds. For the purposes of convenience, compositions according to this paragraph are sometimes referred to herein as Composition 11.

The present invention includes HTCs having a boiling point of from about 50° C. to about 100° C. and a dielectric strength of at least about 40 and which comprise, consist essentially of or consist of one or more Group A2 Compounds. For the purposes of convenience, compositions according to this paragraph are sometimes referred to herein as Composition 12.

The present invention includes methods for removing heat and/or energy from an article, device or fluid or adding heat and/or energy to an article, device or fluid comprising:
(a) providing the article, device or fluid in heat transfer contact with the source of heat and/or energy; and
(b) transferring said heat and/or energy from any composition according to any Compositions 9-12. For the purposes of convenience, methods according to this paragraph are sometimes referred to herein as Heat Transfer Methods 3.

The present invention includes systems and devices that include a heat transfer fluid for transferring heat within and/or to and/or from that system or device, said system and/or device comprising as the heat transfer fluid a composition according to any of Compositions 9-12. For the purposes of convenience, methods according to this paragraph are sometimes referred to herein as System 3.

The present invention includes HTCs that comprise, consists essentially of or consisting of one or more Group A3 Compounds. As the term is used herein, a Group A3 Compound is a compound according to Formula I:

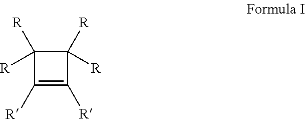

where each R′ is independently selected from H, Cl, and F, where each R is independently selected from H and F, and

provided that the molecule has at least three F substituents. For the purposes of convenience, compositions according to this paragraph are sometimes referred to herein as Composition 13A. The present invention also includes HTCs that comprise, consist essentially of or consist of one or more Group A3 Compounds, provided that no R′ is Cl. Applicants believe that Group A3 Compounds which do not have R′ that is Cl are especially preferred for applications in which low toxicity is highly desirable. For the purposes of convenience, compositions according to this paragraph are sometimes referred to herein as Composition 13B.

The present invention also includes HTCs that comprise, consist essentially of or consist of one or more a Group A3 Compound, provided however, that the composition is essentially free of any Group A3 Compound in which R′ is Cl. For the purposes of convenience, compositions according to this paragraph are sometimes referred to herein as Composition 13C.

The present invention includes HTCs having a boiling point of from about 50° C. to about 100° C. and which comprise, consist essentially of or consist of one or more Group A3 Compounds. For the purposes of convenience, compositions according to this paragraph are sometimes referred to herein as Composition 14A.

The present invention includes HTCs having a boiling point of from about 50° C. to about 100° C. and which comprise, consist essentially of or consist of one or more Group A3 Compounds, provided that no R′ is Cl. For the purposes of convenience, compositions according to this paragraph are sometimes referred to herein as Composition 14B.

The present invention includes HTCs having a boiling point of from about 50° C. to about 100° C. and which comprise, consist essentially of or consist of one or more Group A3 Compounds, provided however, that the composition is essentially free of any Group A3 Compound in which R′ is Cl. For the purposes of convenience, compositions according to this paragraph are sometimes referred to herein as Composition 14C. The present invention includes HTCs having a boiling point of from about 50° C. to about 100° C. and a dielectric constant of less than about 8, and which comprise, consist essentially of or consist of one or more Group A3 Compounds. For the purposes of convenience, compositions according to this paragraph are sometimes referred to herein as Composition 15A.

The present invention includes HTCs having a boiling point of from about 50° C. to about 100° C. and a dielectric constant of less than about 8, and which comprise, consist essentially of or consist of one or more Group A3 Compounds, provided that no R′is Cl. For the purposes of convenience, compositions according to this paragraph are sometimes referred to herein as Composition 15B.

The present invention includes HTCs having a boiling point of from about 50° C. to about 100° C. and a dielectric constant of less than about 8, and which comprise, consist essentially of or consist of one or more Group A3 Compounds, provided however, that the composition is essentially free of any Group A3 Compound in which R′ is Cl. For the purposes of convenience, compositions according to this paragraph are sometimes referred to herein as Composition 15C.

The present invention includes HTCs having a boiling point of from about 50° C. to about 100° C. and a dielectric strength of at least about 40 and which comprise, consist essentially of or consist of one or more Group A3 Compounds. For the purposes of convenience, compositions according to this paragraph are sometimes referred to herein as Composition 16A.

The present invention includes compositions having a boiling point of from about 50° C. to about 100° C. and a dielectric strength of at least about 40, and which comprise, consist essentially of or consist of one or more Group A3 Compounds, provided that no R′is Cl. For the purposes of convenience, compositions according to this paragraph are sometimes referred to herein as Composition 16B.

The present invention includes compositions having a boiling point of from about 50° C. to about 100° C. and a dielectric strength of at least about 40, and which comprise, consist essentially of or consist of one or more Group A3 Compounds, provided however, that the composition is essentially free of any Group A3 Compound in which R′ is Cl. For the purposes of convenience, compositions according to this paragraph are sometimes referred to herein as Composition 16C.

The present invention includes methods for removing heat and/or energy from an article, device or fluid or adding heat and/or energy to an article, device or fluid comprising:
(a) providing the article, device or fluid in heat transfer contact with the source of heat and/or energy; and
(b) transferring said heat and/or energy from any composition according to any Compositions 13-16. For the purposes of convenience, methods according to this paragraph are sometimes referred to herein as Heat Transfer Methods 4.

The present invention includes systems and devices that include a heat transfer fluid for transferring heat within and/or to and/or from that system or device, said system and/or device comprising as the heat transfer fluid a composition according to any of Compositions 13-16. For the purposes of convenience, methods according to this paragraph are sometimes referred to herein as System 4.

The present invention includes HTCs that comprise, consist essentially of or consist of one or more Group A4 Compounds. As the term is used herein, a Group A4 Compound is a compound according to Formula IA:

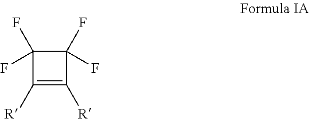

where each R′ is independently selected from F, Cl, OCH₃, OCH₂ (CF₃) and OCH(CF₃)₂. For the purposes of convenience, compositions according to this paragraph are sometimes referred to herein as Composition 17A.

The present invention includes compositions that comprise, consist essentially of or consist of one or more Group A4 Compounds, provided that no R′ is Cl. For the purposes of convenience, compositions according to this paragraph are sometimes referred to herein as Composition 17B.

The present invention includes HTCs having a boiling point of from about 50° C. to about 150° C. and which comprise, consist essentially of or consist of one or more Group A4 Compounds. For the purposes of convenience, compositions according to this paragraph are sometimes referred to herein as Composition 18A.

The present invention includes HTCs having a boiling point of from about 50° C. to about 150° C. and which comprise, consist essentially of or consist of one or more Group A4 Compounds, provided that no R′ is Cl. For the purposes of convenience, compositions according to this paragraph are sometimes referred to herein as Composition 18B.

The present invention includes HTCs having a boiling point of from about 500° C. to about 150° C. and which comprise, consist essentially of or consist of one or more Group A4 Compounds, provided however, that the composition is essentially free of any Group A4 Compound in which R′ is Cl. For the purposes of convenience, compositions according to this paragraph are sometimes referred to herein as Composition 18C.

The present invention includes HTCs having a boiling point of from about 50° C. to about 100° C. and a dielectric constant of less than about 8, and which comprise, consist essentially of or consist of one or more Group A4 Compounds. For the purposes of convenience, compositions according to this paragraph are sometimes referred to herein as Composition 19A.

The present invention includes HTCs having a boiling point of from about 50° C. to about 150° C. and and a dielectric constant of less than about 8, which comprise, consist essentially of or consist of one or more Group A4 Compounds, provided that no R′ is Cl. For the purposes of convenience, compositions according to this paragraph are sometimes referred to herein as Composition 19B.

The present invention includes HTCs having a boiling point of from about 50° C. to about 150° C. and and a dielectric constant of less than about 8, which comprise, consist essentially of or consist of one or more Group A4 Compounds, provided however, that the composition is essentially free of any Group A4 Compound in which R′is Cl. For the purposes of convenience, compositions according to this paragraph are sometimes referred to herein as Composition 19C.

The present invention includes HTCs having a boiling point of from about 50° C. to about 100° C. and a dielectric strength of at least about 40 and which comprise, consist essentially of or consist of one or more Group A4 Compounds. For the purposes of convenience, compositions according to this paragraph are sometimes referred to herein as Composition 20A.

The present invention includes HTCs having a boiling point of from about 50° C. to about 150° C. and and a dielectric strength of at least about 40, which comprise, consist essentially of or consist of one or more Group A4 Compounds, provided that no R′ is Cl. For the purposes of convenience, compositions according to this paragraph are sometimes referred to herein as Composition 20B.

The present invention includes HTCs having a boiling point of from about 50° C. to about 150° C. and and a dielectric strength of at least about 40, which comprise, consist essentially of or consist of one or more Group A4 Compounds, provided however, that the composition is essentially free of any Group A4 Compound in which R′ is Cl. For the purposes of convenience, compositions according to this paragraph are sometimes referred to herein as Composition 20C.

The present invention includes methods for removing heat and/or energy from an article, device or fluid or adding heat and/or energy to an article, device or fluid comprising:
(a) providing the article, device or fluid in heat transfer contact with the source of heat and/or energy; and
(b) transferring said heat and/or energy from any composition according to any Compositions 17-20. For the purposes of convenience, methods according to this paragraph are sometimes referred to herein as Heat Transfer Methods 5.

The present invention includes systems and devices that include a heat transfer fluid for transferring heat within and/or to and/or from that system or device, said system and/or device comprising as the heat transfer fluid a composition according to any of Compositions 17-20. For the purposes of convenience, methods according to this paragraph are sometimes referred to herein as System 5.

The present invention includes HTCs comprising, consisting essentially of or consisting of one or more Group A5 Compounds. As the term is used herein, a Group A5 Compound is a compound according to Formula IA:

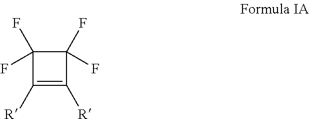

where each R′ is independently selected from F, Cl, OCH₃, OCH₂(CF₃), OCH(CF₃)₂ and OBz, provided than when one R′ is F or Cl, then the other R′ is neither F nor Cl. For the purposes of convenience, compositions according to this paragraph are sometimes referred to herein as Composition 21.

The present invention includes HTCs having a boiling point of from about 100° C. to about 200° C. and which comprise, consist essentially of or consist of one or more Group A5 Compounds. For the purposes of convenience, compositions according to this paragraph are sometimes referred to herein as Composition 22.

The present invention includes HTCs having a boiling point of from about 100° C. to about 200° C. and a dielectric constant of less than about 8, and which comprise, consist essentially of or consist of one or more Group A5 Compounds. For the purposes of convenience, compositions according to this paragraph are sometimes referred to herein as Composition 23.

The present invention includes HTCs having a boiling point of from about 100° C. to about 200° C. and a dielectric strength of at least about 40 and which comprise, consist essentially of or consist of one or more Group A5 Compounds. For the purposes of convenience, compositions according to this paragraph are sometimes referred to herein as Composition 24.

The present invention includes methods for removing heat and/or energy from an article, device or fluid or adding heat and/or energy to an article, device or fluid comprising:
(a) providing the article, device or fluid in heat transfer contact with the source of heat and/or energy; and
(b) transferring said heat and/or energy from any composition according to any Compositions 21-24. For the purposes of convenience, methods according to this paragraph are sometimes referred to herein as Heat Transfer Methods 6.

The present invention includes systems and devices that include a heat transfer fluid for transferring heat within and/or to and/or from that system or device, said system and/or device comprising as the heat transfer fluid a composition according to any of Compositions 21-24. For the purposes of convenience, methods according to this paragraph are sometimes referred to herein as System 6.

The present invention includes HTCs comprising, consisting essentially of or consisting of one or more Group A6 Compounds. As the term is used herein, a Group A6 Compound is a compound according to Formula IA:

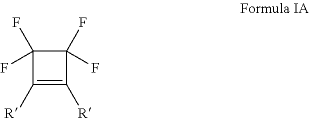

where each R′ is independently selected from Cl, OCH₃, OCH₂(CF₃) and OCH((CF₃))₂, provided than not more than one R′ is Cl.
   For the purposes of convenience, compositions according to this paragraph are sometimes referred to herein as Composition 25.

The present invention includes HTCs having a boiling point of from about 115° C. to about 150° C. and which comprise, consist essentially of or consist of one or more Group A6 Compounds. For the purposes of convenience, compositions according to this paragraph are sometimes referred to herein as Composition 26.

The present invention includes HTCs having a boiling point of from about 115° C. to about 150° C. and a dielectric constant of less than about 8, and which comprise, consist essentially of or consist of one or more Group A6 Compounds. For the purposes of convenience, compositions according to this paragraph are sometimes referred to herein as Composition 27.

The present invention includes HTCs having a boiling point of from about 115° C. to about 150° C. and a dielectric strength of at least about 40 and which comprise, consist essentially of or consist of one or more Group A6 Compounds. For the purposes of convenience, compositions according to this paragraph are sometimes referred to herein as Composition 28

The present invention includes methods for removing heat and/or energy from an article, device or fluid or adding heat and/or energy to an article, device or fluid comprising:
(a) providing the article, device or fluid in heat transfer contact with the source of heat and/or energy; and
(b) transferring said heat and/or energy from any composition according to any Compositions 24-28. For the purposes of convenience, methods according to this paragraph are sometimes referred to herein as Heat Transfer Methods 7.

The present invention includes systems and devices that include a heat transfer fluid for transferring heat within and/or to and/or from that system or device, said system and/or device comprising as the heat transfer fluid a composition according to any of Compositions 24-28. For the purposes of convenience, methods according to this paragraph are sometimes referred to herein as System 7.

The present invention includes novel compounds according to Formula I:

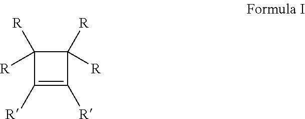

where each R′ is independently selected from F, CF₃, OCH(CF₃)₂ and OBz, where Bz is an unsubstituted benzene,
where each R is independently selected from H, F and CF₃, and
provided that (i) not more than one R can be H; (2) each R′ is the same except when one R′ is F; and (3) when one R′ is F, then the other R′ is CF₃; (4) R′ can be CF3 only when one R′ is F; and (5) R can be CF3 only when an R′ is CF3. For the purposes of convenience, compounds according to this paragraph are sometimes referred to herein as Compound Group B.

The present invention includes novel compounds selected from those depicted in Table B1 below

TABLE B1

| | COMPOUND |
|---|---|
| STRUCTURE* | NUMBER |
| 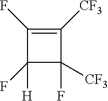 [SMILES: FC1=C(C(F)(F)F)C(F)(C(F)(F)F)C1F] cis-1,3,4-trifluoro-2,3- bis(trifluoromethyl)cyclobut-1-ene | 8A |
| 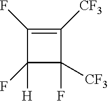 [SMILES: FC1=C(C(F)(F)F)C(F)(C(F)(F)F)C1F] trans-1,3,4-trifluoro-2,3- bis(trifluoromethyl)cyclobut- 1-ene | 8B |
| 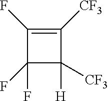 [SMILES: FC1=C(C(F)(F)F)C(C(F)(F)F)C1(F)F] 2,3,3-trifluoro-1,4-bis (trifluoromethyl)cyclobut-1-ene | 9 |
| 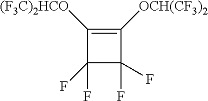 3,3,4,4-pentafluoro-1,2-bis ((1,1,1,3,3,3-hexafluoropropan- 2-yl)oxy)cyclobut-1-ene | 15 |
| 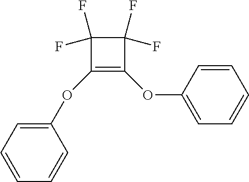 [SMILES: FC1(F)C(Oc2ccccc2)=C(Oc2ccccc2)C1(F)F] ((perfluorocyclobut-1-ene-1,2-diyl) bis(oxy))dibenzene | 17 |
| 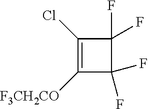 1-chloro-3,3,4,4-tetrafluoro-2- (2,2,2-trifluoroethoxy)cyclobut-1-ene | 18 |

*The compound name is provided for convenience and in the event of an
inconsistency the depicted structure controls.

and for the purposes of convenience, compounds according to this paragraph are sometimes referred to herein as Compound Group B1.

The present invention includes HTCs which comprise, consist essentially of or consist of one or more Group B Compounds. For the purposes of convenience, HTCs according to this paragraph are sometimes referred to herein as Composition 29.

The present invention includes HTCs having a boiling point of from about 25° C. to about 200° C. and which comprise, consist essentially of or consist of one or more Group B Compounds. For the purposes of convenience, HTCs according to this paragraph are sometimes referred to herein as Composition 30.

The present invention includes HTCs having a boiling point of from about 25° C. to about 200° C. and a dielectric constant of less than about 8, and which comprise, consist essentially of or consist of one or more Group B Compounds. For the purposes of convenience, compositions according to this paragraph are sometimes referred to herein as Composition 31.

The present invention includes HTCs having a boiling point of from about 25° C. to about 200° C. and a dielectric strength of at least about 40 and which comprise, consist essentially of or consist of one or more Group B Compounds. For the purposes of convenience, compositions according to this paragraph are sometimes referred to herein as Composition 32.

The present invention includes methods for removing heat and/or energy from an article, device or fluid or adding heat and/or energy to an article, device or fluid comprising:
(a) providing the article, device or fluid in heat transfer contact with the source of heat and/or energy; and
(b) transferring said heat and/or energy from any composition according to any Compositions 29-32. For the purposes of convenience, methods according to this paragraph are sometimes referred to herein as Heat Transfer Methods 8.

The present invention includes systems and devices that include a heat transfer fluid for transferring heat within and/or to and/or from that system or device, said system and/or device comprising as the heat transfer fluid a composition according to any of Compositions 29-32. For the purposes of convenience, methods according to this paragraph are sometimes referred to herein as System 8.

The present invention includes HTCs which comprise, consist essentially of or consist of one or more Group B1 Compounds. For the purposes of convenience, HTCs according to this paragraph are sometimes referred to herein as Composition 33.

The present invention includes HTCs having a boiling point of from about 25° C. to about 200° C. and which comprise, consist essentially of or consist of one or more Group B1 Compounds. For the purposes of convenience, HTCs according to this paragraph are sometimes referred to herein as Composition 34.

The present invention includes HTCs having a boiling point of from about 25° C. to about 200° C. and a dielectric constant of less than about 8, and which comprise, consist essentially of or consist of one or more Group B1 Compounds. For the purposes of convenience, compositions according to this paragraph are sometimes referred to herein as Composition 35.

The present invention includes HTCs having a boiling point of from about 25° C. to about 200° C. and a dielectric strength of at least about 40 and which comprise, consist essentially of or consist of one or more Group B1 Compounds. For the purposes of convenience, compositions according to this paragraph are sometimes referred to herein as Composition 36.

The present invention includes methods for removing heat and/or energy from an article, device or fluid or adding heat and/or energy to an article, device or fluid comprising:
(a) providing the article, device or fluid in heat transfer contact with the source of heat and/or energy; and
(b) transferring said heat and/or energy from any composition according to any Compositions 33-36. For the purposes of convenience, methods according to this paragraph are sometimes referred to herein as Heat Transfer Methods 9.

The present invention includes systems and devices that include a heat transfer fluid for transferring heat within and/or to and/or from that system or device, said system and/or device comprising as the heat transfer fluid a composition according to any of Compositions 33-36. For the purposes of convenience, methods according to this paragraph are sometimes referred to herein as System 9.

The present invention includes heat transfer compositions that comprise or consist essentially of or consist of: (a) any one or more of the compounds in Groups A, A1-A6, B and B1; and (ii) and one or more co-heat transfer components. For the purposes of convenience, compositions according to this paragraph are sometimes referred to herein as Composition 37.

The present invention includes heat transfer compositions that comprise or consist essentially of or consist of: (a) any one or more of the compounds in Groups A, A1-A6, B and B1; and (b) and one or more co-heat transfer components; and (c) a stabilizer and/or a lubricant. For the purposes of convenience, compositions according to this paragraph are sometimes referred to herein as Composition 38.

The present invention includes methods for removing heat and/or energy from an article, device or fluid or adding heat and/or energy to an article, device or fluid comprising:
(a) providing the article, device or fluid in heat transfer contact with the source of heat and/or energy; and
(b) transferring said heat and/or energy from said source to a TMC of the present invention, including each of Compositions 1-38. For the purposes of convenience, methods according to this paragraph are sometimes referred to herein as Heat Transfer Methods 10.

The present invention includes devices and systems for removing heat and/or energy from or adding heat and/or energy to an article, device or fluid comprising at least one vessel, container and/or conduit containing a TMC in thermal contact with said heat and/or energy, wherein said TMC is a TMC of the present invention, including each of Compositions 1-38. For the purposes of convenience, devices and systems according to this paragraph are sometimes referred to herein for convenience as Systems 10.

### BRIEF DESCRIPTION OF THE DRAWINGS

FIG. 1 is a schematic representation of a thermal management system of the present invention.

FIG. 2A is a schematic representation of a first exemplary immersion cooling system according to the present invention.

FIG. 2B is a schematic representation of a second exemplary immersion cooling system according to the present invention.

FIG. 3 is a schematic illustration of a battery thermal management system according to one embodiment of the present invention.

FIG. 4 is a photograph showing a battery thermal management system according to one embodiment of the present invention.

FIG. 5 is a schematic diagram of an exemplary organic Rankine cycle.

FIG. 6 is a schematic diagram of an exemplary heat pump.

FIG. 7 is a schematic diagram of an exemplary secondary loop system.

### DETAILED DESCRIPTION

### Definitions

As used herein, the following terms have the meanings indicated below unless specifically indicated otherwise.

The term Heat Transfer Composition and related word forms, means a composition in the form of a fluid (liquid or gas) which is used to transfer heat or energy from one fluid, article or device to another location, and thus includes for example refrigerants, temperature control fluids and working fluids for Rankine cycles.

When a heat transfer composition is used in thermal management to keep an device or article within a particular temperature range (e.g., in electronic cooling), it is sometimes referred herein as a thermal management fluid.

The component(s) that are present in a heat transfer composition for the purpose of transferring heat (as opposed to, for example, providing lubrication or stabilization) in a heat transfer system (e.g. a vapour compression heat transfer system), that component or combination of components are sometimes referred to herein as a refrigerant.

Device means an article, object or contrivance which is heated, cooled, or maintained at a predetermined temperature.

Operating Electronic Device, and related word forms, means a device, or a component of a device, which is in the process of performing its intended function by receiving, and/or transmitting and/or producing electrical energy and/or electronic signals. Thus, the term “operating electronic device” as used herein includes, for example, a battery which is in the process of providing a source of electrical energy to another component and also a battery which is being charged or recharged.

Thermal contact, and related forms thereof, includes direct contact with the surface and indirect contact though another body or fluid which facilitates the flow of heat between the surface and the fluid.

Global Warming Potential (“GWP”) was developed to allow comparisons of the global warming impact of different gases. It is a measure of how much energy the emission of one ton of a gas will absorb over a given period of time, relative to the emission of one ton of carbon dioxide. The larger GWP, the more that a given gas warms the Earth compared to CO2 over that time period. The time period usually used for GWP is 100 years. GWP provides a common measure, which allows analysts to add up emission estimates of different gases.

LC₅₀ is a measure of the acute toxicity of a compound. The acute inhalation toxicity of a compound can be assessed using the method described in the OECD Guideline for Testing of Chemicals No. 403 “Acute Inhalation Toxicity” (2009), Method B.2. (Inhalation) of Commission Regulation (EC) No. 440/2008.

Flash Point refers the lowest temperature at which vapors of the liquid will keep burning after the ignition source is removed as determined in accordance with ASTM D3828.

Non-flammable in the context of a thermal management composition or fluid means compounds or compositions which are determined to be non-flammable. The flash point of a thermal management composition or fluid refers the lowest temperature at which vapors of the composition will keep burning after the ignition source is removed as determined in accordance with ASTM D3828. Thermal management compositions or fluids which do not have a flash point below 100° F. (37.8° C.) are classified as “non-flammable” in accordance with NFPA 30: Flammable and Combustible Liquid Code. for liquids means fluids which do not have a flash point below 100° F. (37.8° C.) are classified as “non-flammable” in accordance with NFPA 30: Flammable and Combustible Liquid Code.

In the context of a refrigerant composition, a compound or composition which is non-flammable and low or no-toxicity would be classified as “A1” by ASHRAE Standard 34-2016 Designation and Safety Classification of Refrigerants and described in Appendix B1 to ASHRAE Standard 34-2016.

No or low toxicity means a fluid classified as class “A” by ASHRAE Standard 34-2016 Designation and Safety Classification of Refrigerants and described in Appendix B1 to ASHRAE Standard 34-2016.

Capacity is the amount of cooling provided, in BTUs/hr, by the refrigerant in the refrigeration system. This is experimentally determined by multiplying the change in enthalpy in BTU/lb, of the refrigerant as it passes through the evaporator by the mass flow rate of the refrigerant. The enthalpy can be determined from the measurement of the pressure and temperature of the refrigerant. The capacity of the refrigeration system relates to the ability to maintain an area to be cooled at a specific temperature. The capacity of a refrigerant represents the amount of cooling or heating that it provides and provides some measure of the capability of a compressor to pump quantities of heat for a given volumetric flow rate of refrigerant. In other words, given a specific compressor, a refrigerant with a higher capacity will deliver more cooling or heating power.

Coefficient of Performance (hereinafter “COP”) is a universally accepted measure of refrigerant performance, especially useful in representing the relative thermodynamic efficiency of a refrigerant in a specific heating or cooling cycle involving evaporation or condensation of the refrigerant. In refrigeration engineering, this term expresses the ratio of useful refrigeration or cooling capacity to the energy applied by the compressor in compressing the vapor and therefore expresses the capability of a given compressor to pump quantities of heat for a given volumetric flow rate of a heat transfer fluid, such as a refrigerant. In other words, given a specific compressor, a refrigerant with a higher COP will deliver more cooling or heating power. One means for estimating COP of a refrigerant at specific operating conditions is from the thermodynamic properties of the refrigerant using standard refrigeration cycle analysis techniques (see for example, R. C. Downing, FLUOROCARBON REFRIGERANTS HANDBOOK, Chapter 3, Prentice-Hall, 1988 which is incorporated herein by reference in its entirety).

Thermal Efficiency is a measure of how efficiently one can convert energy from a heat source to work. This property is generally used to characterize the performance of an Organic Rankine Cycle System much like COP is used to measure the efficiency of a vapor compression system. One means for estimating COP of a refrigerant at specific operating conditions is from the thermodynamic properties of the refrigerant using standard refrigeration cycle analysis techniques (see for example, Engineering and Chemical Thermodynamics, Milo D. Koretsky. Wiley 2004, page 138.

As used herein, the singular forms “a”, “an”, and “the” include plural referents unless the content clearly dictates otherwise. As used in this specification, the term “or” is generally employed in its sense including “and/or” unless the content clearly dictates otherwise.

As used herein, the recitation of numerical ranges by endpoints includes all numbers subsumed within that range (e.g. 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

### Compounds

As indicated above, the present invention includes novel compounds according to Formula I:

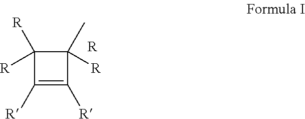

where each R′ is independently selected from F, CF₃, OCH₃, OCH(CF₃)₂ and OBz, provided that each R′ is the same except when one R′ is F, then the other R′ is not F
where Bz is an unsubstituted benzene,
where each R is independently selected from H, F and CF₃, and
provided that the molecule has at least three F substituents on the cyclobutene ring. Compounds according to the present invention can be formed by those skilled in the art based on the teaching contained herein, including specifically as disclosed in the Examples.

One novel compound in accordance with the present invention is:

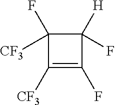 [SMILES: FC1=C(C(F)(F)F)C(F)(C(F)(F)F)C1F]

Another novel compound in accordance with the present invention is:

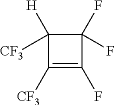 [SMILES: FC1=C(C(F)(F)F)C(C(F)(F)F)C1(F)F]

Another novel compound in accordance with the present invention is:

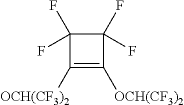

Another novel compound in accordance with the present invention is:

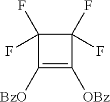

The novel compounds of the present invention have one or more of the following properties, and preferably at least two of the physical properties:

| | Broad | Intermediate | Narrow |
|---|---|---|---|
| | Range | Range | Range |
| Property | | | |
| Boiling Point, | 50-250 | 50-200 | 50-150 |
| ° C. | | | |
| Kinematic | 0.3-1.2 | 0.3-1.1 | 0.4-1.1 |
| Viscosity, cSt | | | |
| Dielectric | 1.5-10 | 1.5-5 | 1.5-3 |
| Constant at | | | |
| 1 kHz | | | |
| Dielectric | >30 | >40 | 30-80 |
| Strength, 0.1″ | | | |
| gap, KV | | | |
| Thermal | >0.055 | >0.065 | 0.055-0.2 |
| Conductivity, | | | |
| W/m-K | | | |

The following compounds of the present invention have the following properties:

| | | Normal | Miscibility | | Heat of |
|---|---|---|---|---|---|
| | Compound | Boiling | with | Flash | Vaporization, |
| 2. Compound | No. | Point, ° C. | mineral oil | Point | (kcal/mol) |
| 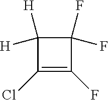 1-chloro-2,3,3- trifluorocyclobut-1-ene | 1 | 52 | =/>1:1 | No flash point | |
| 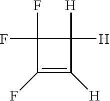 1,4,4-trifluorocyclobut- 1-ene | 2 | 32 | Not miscible | Flash point observed | |
| 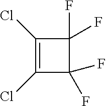 1,2-dichloro-3,3,4,4- tetrafluorocyclobut-1-ene | 3 | 58 | =/>1:1 | No flash point | |
| 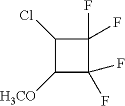 [SMILES: COC1C(Cl)C(F)(F)C1(F)F] 1-chloro-3,3,4,4-tetrafluoro- 2-methoxycyclobut-1-ene | 4 | 110 | | | |
| 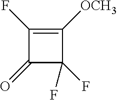 2,4,4-trifluoro-3- methoxycyclobut-2-en-1-one | 5 | | | | |
| 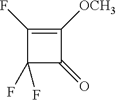 3,4,4-trifluoro-2- methoxycyclobut-2-en-1-one | 6 | | | | |
| 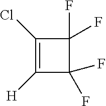 1-chloro-3,3,4,4- tetrafluorocyclobut-1-ene | 7 | 55 | | | |
| 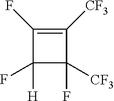 [SMILES: FC1=C(C(F)(F)F)C(F)(C(F)(F)F)C1F] cis-1,3,4-trifluoro-2,3-bis (trifluoromethyl)cyclobut-1-ene | 8A | | | | |
| 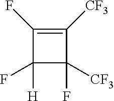 [SMILES: FC1=C(C(F)(F)F)C(F)(C(F)(F)F)C1F] trans-1,3,4-trifluoro-2,3-bis (trifluoromethyl)cyclobut-1-ene | 8B | | | | |
| 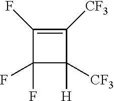 [SMILES: FC1=C(C(F)(F)F)C(C(F)(F)F)C1(F)F] 2,3,3-trifluoro-1,4-bis (trifluoromethyl)cyclobut-1-ene | 9 | | | | |
| 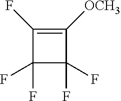 [SMILES: COC1=C(F)C(F)(F)C1(F)F] 1,3,3,4,4-pentafluoro-2- methoxycyclobut-1-ene | 10 | 87 | | | |
| 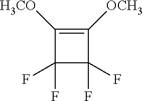 [SMILES: COC1=C(OC)C(F)(F)C1(F)F] 3,3,4,4-tetrafluoro-1,2- dimethoxycyclobut-1-ene | 11 | 127 | | | |
| 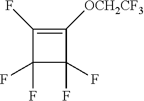 1,3,3,4,4-pentafluoro-2- (trifluoromethoxy)cyclobut-1-ene | 12 | 95 | | | |
| 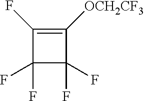 3,3,4,4-pentafluoro-1,2- bis(trifluoromethoxy)cyclobut-1-ene | 13 | 144 | | | 32 |
| 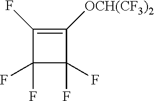 1,3,3,4,4-pentafluoro-2-((1,1,1, 3,3,3-hexafluoropropan-2- yl)oxy)cyclobut-1-ene | 14 | 81 | | | 31 |
| 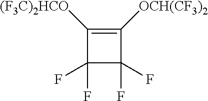 3,3,4,4-pentafluoro-1,2- bis((1,1,1,3,3,3-hexafluoropropan- 2-yl)oxy)cyclobut-1-ene | 15 | 126-127 | | | 34 |
| 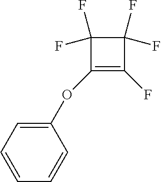 [SMILES: FC1=C(Oc2ccccc2)C(F)(F)C1(F)F] ((perfluorocyclobut-1- en-1-yl)oxy)benzene | 16 | | | | 42 |
| 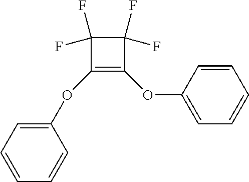 [SMILES: FC1(F)C(Oc2ccccc2)=C(Oc2ccccc2)C1(F)F] ((perfluorocyclobut-1-ene-1,2- diyl)bis(oxy))dibenzene | 17 | | | | 75 |
| 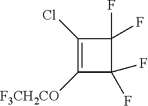 1-chloro-3,3,4,4-trifluoro-2- (2,2,2-trifluoroethoxy) cyclobut-1-ene | 18 | 118-119 | | | 38 |

In addition, the compounds of the present invention have good miscibility with lubricants, including lubricants used in vapour compression refrigeration systems. For example, the Compounds 1 and 3 are fully miscible with e mineral oil at concentrations up to about 66.7 weight percent as indicated in the following table.

| Compound | Compound No. | Miscibility |
|---|---|---|
| 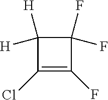 1-chloro-2,3,3- trifluorocyclobut-1-ene | 1 | Miscible up to 66.7% |
| 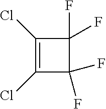 1,2-dichloro-3,3,4,4- tetrafluorocyclobut-1-ene | 3 | Miscible up to 66.7% |

### Heat Transfer Compositions

The present invention provides various methods, processes and uses of the heat transfer compositions of the present invention, including each of Compositions 1-38, are fluids (i.e., liquids and/or gases) that may be used to transmit heat from one location to another (or from one body or article to another). For example, the heat transfer compositions may be used to keep the temperature of a device below a defined upper and/or above a defined lower temperature. In another example, the heat transfer compositions may be used for energy conversion, as in the capture of waste heat from industrial or other processes and the conversion to electrical or mechanical energy.

The heat transfer fluids of the present invention comprise a heat transfer composition of the present invention, including each of Compositions 1-4, wherein Group A Compound(s) are present in the composition in an amount of at least about 50% by weight, or at least about 70% by weight, or at least about 90% by weight or at least about 95% by weight or at least about 99% by weight, excluding non-heat transfer components, or the heat transfer fluid may consist essentially of or consist of Group A Compound(s).

The heat transfer fluids of the present invention comprise a heat transfer composition of the present invention, including each of Compositions 5-8, wherein Group A1 Compound(s) are present in the composition in an amount of at least about 50% by weight, or at least about 70% by weight, or at least about 90% by weight or at least about 95% by weight or at least about 99% by weight, excluding non-heat transfer components, or the heat transfer fluid may consist essentially of or consist of Group A1 Compound(s).

The heat transfer fluids of the present invention comprise a heat transfer composition of the present invention, including each of Compositions 9-12, wherein Group A2 Compound(s) are present in the composition in an amount of at least about 50% by weight, or at least about 70% by weight, or at least about 90% by weight or at least about 95% by weight or at least about 99% by weight, excluding non-heat transfer components, or the heat transfer fluid may consist essentially of or consist of Group A2 Compound(s).

The heat transfer fluids of the present invention comprise a heat transfer composition of the present invention, including each of Compositions 13-16, wherein Group A3 Compound(s) are present in the composition in an amount of at least about 50% by weight, or at least about 70% by weight, or at least about 90% by weight or at least about 95% by weight or at least about 99% by weight, excluding non-heat transfer components, or the heat transfer fluid may consist essentially of or consist of Group A3 Compound(s).

The heat transfer fluids of the present invention comprise a heat transfer composition of the present invention, including each of Compositions 17-20, wherein Group A4 Compound(s) are present in the composition in an amount of at least about 50% by weight, or at least about 70% by weight, or at least about 90% by weight or at least about 95% by weight or at least about 99% by weight, excluding non-heat transfer components, or the heat transfer fluid may consist essentially of or consist of Group A4 Compound(s).

The heat transfer fluids of the present invention comprise a heat transfer composition of the present invention, including each of Compositions 21-24, wherein Group A5 Compound(s) are present in the composition in an amount of at least about 50% by weight, or at least about 70% by weight, or at least about 90% by weight or at least about 95% by weight or at least about 99% by weight, excluding non-heat transfer components, or the heat transfer fluid may consist essentially of or consist of Group A5 Compound(s).

The heat transfer fluids of the present invention comprise a heat transfer composition of the present invention, including each of Compositions 25-28, wherein Group A6 Compound(s) are present in the composition in an amount of at least about 50% by weight, or at least about 70% by weight, or at least about 90% by weight or at least about 95% by weight or at least about 99% by weight, excluding non-heat transfer components, or the heat transfer fluid may consist essentially of or consist of Group A6 Compound(s).

The heat transfer fluids of the present invention comprise a heat transfer composition of the present invention, including each of Compositions 29-32, wherein Group B Compound(s) are present in the composition in an amount of at least about 50% by weight, or at least about 70% by weight, or at least about 90% by weight or at least about 95% by weight or at least about 99% by weight, excluding non-heat transfer components, or the heat transfer fluid may consist essentially of or consist of Group B Compound(s).

The heat transfer fluids of the present invention comprise a heat transfer composition of the present invention, including each of Compositions 33-36, wherein Group B1 Compound(s) are present in the composition in an amount of at least about 50% by weight, or at least about 70% by weight, or at least about 90% by weight or at least about 95% by weight or at least about 99% by weight, excluding non-heat transfer components, or the heat transfer fluid may consist essentially of or consist of Group B1 Compound(s).

The heat transfer fluids of the present invention comprise a heat transfer composition of the present invention, including each composition within Compositions 37 and 38, wherein compound(s) (a) of the present invention are as defined in according to Compositions 37A and 37B respectively and co-heat transfer component(s) (b) are present in the composition are as defined in according to Compositions 37A and 37B, in amounts as indicated in the Table below based on the total weight of the heat transfer components in the composition:

| Composition | Wt % Compound(s) of the | Wt % of Co-heat transfer |
|---|---|---|
| No. | invention | components |
| 37A and 38A | 1-99 | 99-1 |
| 37B and 38B | 1-10 | 90-99 |
| 37C and 38C | 10-20 | 80-90 |
| 37D and 38D | 20-30 | 70-80 |
| 37E and 38E | 30-40 | 60-70 |
| 37F and 38F | 40-50 | 50-40 |
| 37G and 38G | 50-60 | 40-60 |
| 37H and 38H | 60-70 | 30-40 |
| 37I and 38I | 70-80 | 20-30 |
| 37J and 38J | 80-90 | 10-20 |

Any reference herein to Composition 37 or Composition 38 is understood that mean and include each of the compositions referenced herein by those numbers with a suffix letter, as in the above table with suffixes A through J and is in the suffix letters K through T_below, unless the context clearly indicates otherwise.

The present invention includes HTCs, including each Composition 37 and Composition 38, having a boiling point of from about 25° C. to about 200° C. Compositions according to this paragraph are referred to for convenience as Composition 37K and Composition 38K, respectively.

The present invention includes HTCs, including each Composition 37 and Composition 38, having a boiling point of from about 25° C. to about 150° C. Compositions according to this paragraph are referred to for convenience as Composition 37L and Composition 38M, respectively,

The present invention includes HTCs, including each Composition 37 and Composition 38, having a boiling point of from about 50° C. to about 100° C. Compositions according to this paragraph are referred to for convenience as Composition 37N and Composition 38N, respectively.

The present invention includes HTCs, including each Composition 37 and Composition 38, having a boiling point of from about 100° C. to about 150° C. Compositions according to this paragraph are referred to for convenience as Composition 37O and Composition 38O, respectively.

The present invention includes HTCs, including each Composition 37 and Composition 38, having a boiling point of from about 115° C. to about 150° C. Compositions according to this paragraph are referred to for convenience as Composition 37P and Composition 38P, respectively.

The present invention includes HTCs, including each Composition 37 and Composition 38, having a boiling point of from about 100° C. to about 200° C. Compositions according to this paragraph are referred to for convenience as Composition 37Q and Composition 38Q, respectively.

The present invention includes HTCs, including each Composition 37 and Composition 38, having a boiling point of from about 150° C. to about 200° C. Compositions according to this paragraph are referred to for convenience as Composition 37R and Composition 38R, respectively.

The present invention includes HTCs, including each Composition 37 and Composition 38, having a dielectric constant of less than about 8. Compositions according to this paragraph are referred to for convenience as Composition 37S and Composition 38S, respectively.

The present invention includes HTCs, including each Composition 37 and Composition 38, having a dielectric strength of greater than 40. Compositions according to this paragraph are referred to for convenience as Composition 37T and Composition 38T, respectively.

The present invention includes heat transfer compositions in accordance with each composition herein within the definition of Composition 37 and Composition 38 in which the co-heat transfer composition is selected from the group consisting of HFE-7000, HFE-7200, HFE-7100, HFE-7300, HFE-7500, HFE-7600, trans-1,2-dichloroethylene, n-pentane, cyclopentane, ethanol, perfluoro (2-methyl-3-pentanone) (Novec 1230), cis-HFO-1336mzz, trans-HFO-1336mzz, HF-1234yf, HFO-1234ze(E), HFO-1233zd(E), HFO-1233zd(Z) and combinations of any two or more of these.

The heat transfer composition (and therefore also the thermal management fluid, working fluid or refrigerant) of the present invention, including each of Compositions 1-38, has a low GWP, including a GWP of not greater than about 1000, or not greater than about 700, or not greater than about 500, or not greater than about 300, or not greater than about 150.

Preferably, the heat transfer composition of the present invention, including each of Compositions 38, comprises a lubricant. The lubricant lubricates the refrigeration compressor using the refrigerant. The lubricant may be present in the heat transfer composition in amounts of from about 5% to about 30% by weight of heat transfer composition. Lubricants such as Polyol Esters (POEs), Poly Alkylene Glycols (PAGs), PAG oils, polyvinyl ethers (PVEs), and poly(alpha-olefin) (PAO) and combinations thereof may be used in the heat transfer compositions of the present invention.

Preferred lubricants include POEs and PVEs, more preferably POEs. Of course, different mixtures of different types of lubricants may be used. For example, the lubricant may be a PAG if the refrigerant is used in mobile air conditioning applications.

The heat transfer composition therefore comprises a refrigerant of the invention and a lubricant selected from a POE, a PAG or a PVE.

The heat transfer composition of the present invention may consist essentially of or consist of a heat transfer fluid and lubricant as described above.

Commercially available mineral oils include Witco LP 250 (registered trademark) from Witco, Zerol 300 (registered trademark) from Shrieve Chemical, Sunisco 3GS from Witco, and Calumet R015 from Calumet. Commercially available alkyl benzene lubricants include Zerol 150 (registered trademark). Commercially available esters include neopentyl glycol dipelargonate, which is available as Emery 2917 (registered trademark) and Hatcol 2370 (registered trademark). Other useful esters include phosphate esters, dibasic acid esters, and fluoroesters.

The heat transfer composition may include a compatibilizer for the purpose of aiding compatibility and/or solubility of the lubricant. Suitable compatibilizers may include propane, butanes, pentanes, and/or hexanes. When present, the compatibilizer is preferably present in an amount of from about 0.5% to about 5% by weight of the heat transfer composition. Combinations of surfactants and solubilizing agents may also be added to the present compositions to aid oil solubility, as disclosed by U.S. Pat. No. 6,516,837, the disclosure of which is incorporated by reference.

### Uses, Methods, Systems and Devices

The present invention includes method for transferring heat as described herein, included methods as specifically described above and hereinafter.

The present invention also includes devices and systems for transferring heat as described herein, included devices and systems as specifically described above and hereinafter.

The heat transfer fluid, thermal management fluid, refrigerant, working fluid and heat transfer compositions, including each of Compositions 1-38, of the invention are provided for use for heating and/or cooling as set out below.

Thus, the present invention describes a method of heating or cooling a fluid or body using a heat transfer fluid, thermal management fluid, refrigerant, working fluid or heat transfer compositions of the invention, including each of Compositions 1-38.

### Thermal Management Methods, Devices, Systems and Uses

In nearly every modern application of electronics, the dissipation of heat is an important consideration. For example, in portable and hand-held devices, the desire to miniaturize while adding functionality increases the thermal power density, which increases the challenge of cooling the electronics within them. As computational power increases within desktop computers, datacenters and telecommunications centers, so does the heat output. Power electronic devices such as the traction inverters in plug-in electric or hybrid vehicles, wind turbines, train engines, generators and various industrial processes make use of transistors that operate at ever higher currents and heat fluxes.

As discussed above, when a heat transfer fluid of the present invention, including each of Compositions 1-38, is used in a method or device or system of cooling and/or heating in an electronic device, it is sometimes referred to herein as a thermal management fluid. The thermal management fluid therefore corresponds to the heat transfer fluid as discussed in this application.

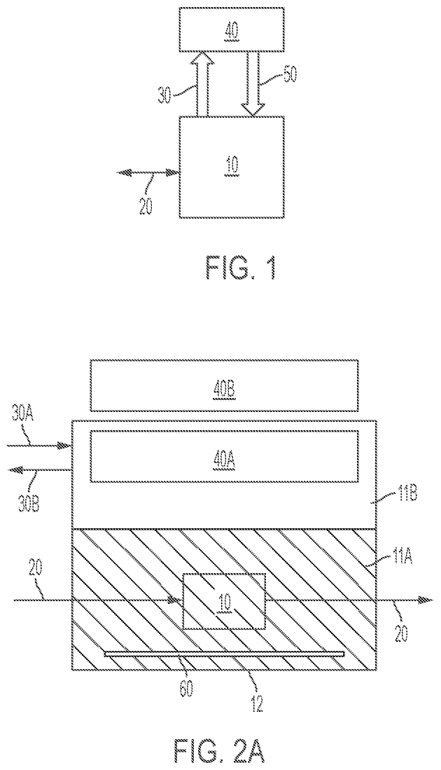

Preferred embodiments of the present thermal management methods, including Heat Transfer Methods 1, will now be described in connection with FIG. 1 in which an operating electronic device is shown schematically as 10 having a source of electrical energy and/or signals 20 flowing into and/or out of the device 10 and which generates heat as a result of its operation based on the electrical energy and/or signals 20. The thermal management fluid of the present invention is provided in thermal contact with the operating device 10 such that it removes heat, represented by the out flowing arrow 30. Heat is removed from the operating electronic device by sensible heat being added to the liquid thermal management fluid of the present invention (i.e., increasing the temperature of the liquid), or by causing a phase change in the thermal management liquid (i.e., vaporizing the liquid) or a combination of these. In preferred embodiments, the methods provide a supply of heat transfer fluid of the present invention, including each of Compositions 1-38, to the device 10 such that the flow of heat from the device 10 through the present heat transfer fluid 30 maintains the operating electrical device at or within a preferred operating temperature range. In preferred embodiments, the preferred operating temperature range of the electrical device is from about 70° C. to about 150° C., and even more preferably from about 70° C. to about 120° C., and the flow of heat 30 from the device 10 through the present heat transfer fluid energy maintains the operating electrical device at or within such preferred temperature ranges. Preferably, the heat transfer fluid 30 of the present invention, which has absorbed heat from the device, is in thermal contact with a heat sink, represented schematically as 40, at a temperature below the temperature of the heat transfer fluid 30 and thereby transfers the heat generated by the device 10 to the heat sink 40. In this way, the heat-depleted heat transfer fluid of the present invention 50 can be returned to the electronic device 10 to repeat the cycle of cooling.

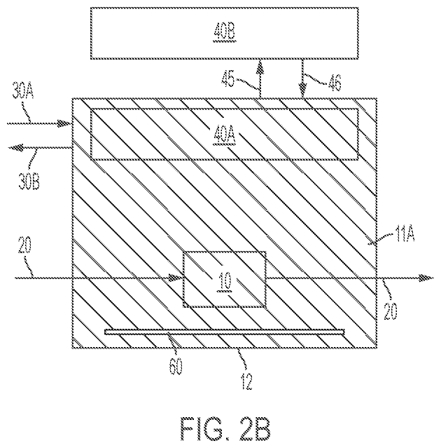

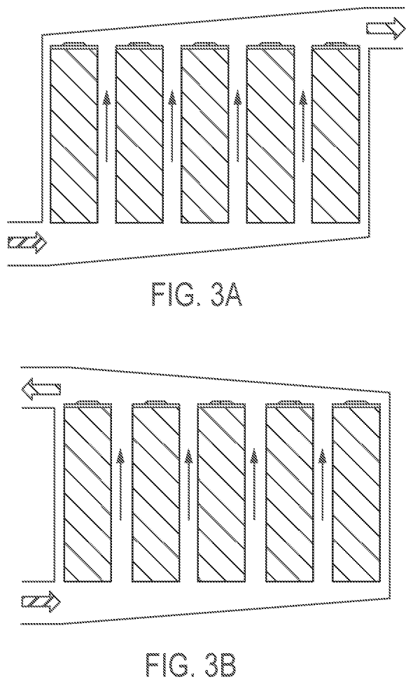

In a preferred embodiment of the present methods, the step of removing heat through a heat transfer composition of the present invention, including each of Compositions 1-38, comprises evaporating the heat transfer composition of the present invention using the heat generated by the operation of the electronic device, and the step of transferring that heat from the heat transfer composition to the heat sink comprises condensing the heat transfer fluid by rejecting heat to the heat sink. In such methods, the temperature of the heat transfer fluid of the present invention, including each of Compositions 1-38, during said evaporation step is preferably greater than 50° C., or preferably greater than about 55° C., or preferably in the range of from about 55° C. to about 85° C., or preferably from about 65° C. to about 75° C. Applicants have found that the present heat transfer fluids, including each of Compositions 1-38, provide excellent performance in such methods and at the same time allow the use of relatively low cost, lightweight and reliable equipment to provide the necessary cooling, as will be explained further in connection with particular embodiments as described in connection with FIG. 2A below.

In a further preferred embodiment of the present methods, the step of removing heat through the present heat transfer composition, including each of Compositions 1-38, comprises adding sensible heat to the liquid heat transfer composition of the present invention (e.g., raising the temperature of the liquid up to about 70° C. or less at about atmospheric pressure, i.e., wherein the fluid is not required to be in a high pressure container or vessel) using the heat generated by the operation of the electronic device, and the step of transferring that heat from the heat transfer composition to a heat sink and thereby reducing the liquid temperature by rejecting heat to the heat sink. The cooled liquid is then returned to thermal contact with the electrical device wherein the cycle starts over. In preferred embodiments, the temperature of the heat transfer liquid that is used to transfer heat to the heat sink is greater than about 40° C., or preferably greater than about 55° C., or preferably in the range of from about 45° C. to about 70° C., or preferably from about 45° C. to about 65° C., and preferably is at a pressure that is about atmospheric. Applicants have found that the present heat transfer liquids, including each of Compositions 1 -38, provide excellent performance in such methods and at the same time allow the use for relatively low cost, lightweight and reliable equipment to provide the necessary cooling, as will be explained further in connection with particular embodiments as described in connection with FIG. 2B below.

It will be appreciated by those skilled in the art that the present invention comprises systems and methods which use both sensible heat transfer and phase change heat transfer as describe above.

A particular method according to the present invention will now be described in connection with FIGS. 2A and 2B in which an electronic device 10 is contained in an appropriate container 12, and preferably a sealed container, and is in direct contact with, and preferably fully immersed in liquid heat transfer composition of the present invention 11A (shown schematically by gray shading), including each of Compositions 1-38. The operating electronic device 10 has a source of electrical energy and/or signals 20 flowing into and/or out of the container 12 and into and/or out of device 10, which generates heat as a result of its operation based on the electrical energy and/or signals 20. As those skilled in the art will appreciate, it is a significant challenge to discover a heat transfer fluid that can perform effectively in such applications since the fluid must not only provide all of the other properties mentioned above, it must be able to do so while in intimate contact with an operating electronic device, that is, one which involves the flow of electrical current/signals. It will be appreciated that many fluids that might be otherwise viable for use in such applications will not be useable because they will either short-out the device, degrade when exposed to the conditions created by the operation of the electronic device or have some other property detrimental to operation when in contact with an operating electronic device.

In contrast, the present methods produce excellent results by providing the thermal management fluid of the present invention, including each of Compositions 1-38, in direct thermal and physical contact with the device 10 as it is operating. This heat of operation is safely and effectively transferred to the thermal management fluid 11A, including each of Compositions 1-38, by: (a) causing the liquid phase of the fluid to evaporate and form vapor 11B; or (b) raising the temperature of the liquid thermal management fluid 11A; or (c) a combination of (a) and (b).

In the case of the phase change heat transfer systems of the present invention, reference is made herein to FIG. 2A. In such an operation, heat is carried away from the device 10 as the liquid evaporates and the vapor rises through the remaining thermal management liquid, including each of Compositions 1-38, in the container 12. The thermal management fluid vapor 11B then rejects the heat it has absorbed to a heat sink 40, which can be an enclosed heat sink 40A and/or an external heat sink 40B. An example of a heat sink that is internal to the container 12 are condenser coils 30A and 30B with circulating liquid, such as water, at a temperature below the condensing temperature of the thermal management fluid vapor. An example of a heat sink that is external to the container 12 would be passing relatively cool ambient air over the container 12 (which preferably in such case include cooling fins or the like), which will serve to condense the heat transfer vapor 11B on the interior surface of the container. As a result of this condensation, liquid thermal management fluid is returned to the pool of liquid fluid 11A in which the device 10 remains immersed in operation.

In the case of a sensible heat transfer systems of the present invention, reference is made herein to FIG. 2B. In such an operation heat is carried away from the device as the temperature of liquid 11A,—including each of Compositions 1-38, increases upon accepting heat being generated by the device, which is immersed, and preferably substantially fully immersed in the thermal management fluid 11A of the present invention. The higher temperature thermal management fluid liquid 11A then rejects the heat it has absorbed to a heat sink 40, which can be an enclosed heat sink 40A and/or an external heat sink 40B. An example of a heat sink that is internal to the container 12 are cooling coils 30A and 30B with circulating liquid, such as water, at a temperature below the temperature of heated liquid. An example of a heat sink that is external to the container 12 would be removing heated liquid 11A from the container through a conduit 45 where it is thermally contacted with a cool fluid, such as might be provided by relatively cool ambient air or a refrigerant, which will serve to lower the temperature of the liquid. Cooled liquid is then returned via conduit 46.

Optionally, but preferably in certain embodiments involving thermal management of the batteries used in electronic vehicles, the thermal management system includes a heating element which is able to heat the thermal management fluid, including each of Compositions 1-38, such as for example an electrical heating element 60 which is also immersed in the thermal management fluid. As those skilled in the art will appreciate, the batteries in electronic vehicles (which would correspond to the operating electronic device 10 in FIGS. 2A and 2B) can reach relatively low temperatures while parked outside in the winter months in many geographical locations, and frequently such low temperature conditions are not desirable for battery operation. Accordingly, the thermal management system of the present invention can include sensors and control modules (not shown) which turn on the heating element when the battery temperature is below a predetermined level. In such a case, the heater 60 would be activated, the thermal management liquid 11A would be heated, and would in turn transfer this heat to the electronic device 10 until the minimum temperature is reached. Thereafter during operation, the thermal management fluid of the present invention, including each of Compositions 1-38, would serve the cooling function as described above.

For the purposes of this invention, the thermal management fluid, including each of Compositions 1-38, can be in direct contact with the heat-generating component or in indirect contact with the heat-generating component.

When the thermal management fluid, including each of Compositions 1-38, is in indirect contact with the heat-generating component, the thermal management fluid can be used in a closed system in the electronic device, which may include at least two heat exchangers. When the thermal management fluid is used to cool the heat-generating component, heat can be transferred from the component to the thermal management fluid, usually through a heat exchanger in contact with at least a part of the component or the heat can be transferred to circulating air which can conduct the heat to a heat exchanger that is in thermal contact with the thermal management fluid.

In a particularly preferred feature of the present invention, the thermal management fluid, including each of Compositions 1-38, is in direct contact with the heat-generating component. In particular, the heat generating component is fully or partially immersed in the thermal management fluid. Preferably the heat generating component is fully immersed in the thermal management fluid. The thermal management fluid, as a warmed fluid or as a vapor, can then be circulated to a heat exchanger which takes the heat from the fluid or vapor and transfers it to the outside environment. After this heat transfer, the cooled thermal management fluid (cooled or condensed) is recycled back into the system to cool the heat-generating component.

When the thermal management fluid is a single-phase liquid, it will remain liquid when heated by the heat-generating component. Thus, the thermal management fluid can be brought into contact with the heat generating component, resulting in the removal of the heat from the heat generating component and the production of a thermal management fluid with a higher temperature. The thermal management fluid is then transported to a secondary cooling loop, such as a radiator or another refrigerated system. An example of such a system is illustrated in FIG. 2, where the thermal management fluid enters a battery pack enclosure containing a number of cells and exits the enclosure having taken up heat from the battery pack.

When the thermal management fluid of the present invention, including each of Compositions 1-38, is present in two phases, the heat-generating component is in thermal contact with the thermal management fluid and transfers heat to the thermal management fluid, resulting in the boiling of the thermal management fluid. The thermal management fluid is then condensed. An example of such a system is where the heat-generating component is immersed in the thermal management fluid and an external cooling circuit condenses the boiling fluid into a liquid state.

Electrical conductivity and/or dielectric strength of a thermal management fluid becomes important if the fluid comes in direct contact with the electronic components of the electronic device (such as in direct immersion cooling), or if the thermal management fluid leaks out of a cooling loop or is spilled during maintenance and comes in contact with the electrical circuits. Thus, the thermal management fluid of the present invention, including each of Compositions 1-38, is preferably an electrically insulating thermal management fluid.

The thermal management fluid of the present invention, including each of Compositions 1-38, may be recirculated passively or actively in the device, for example by using mechanical equipment such as a pump. In a preferred feature of the present invention, the thermal management fluid of the present invention, including each of Compositions 1-38, is recirculated passively in the device.

Passive recirculating systems work by transferring heat from the heat-generating component to the thermal management fluid until it typically is vaporized, allowing the heated vapor to proceed to a heat exchange surface at which it transfers its heat to the heat exchanger surface and condenses back into a liquid. It will be appreciated that the heat exchange surface can be part of a separate heat exchange unit and/or can be integral with the container, as described above for example in connection with FIG. 2. The condensed liquid then returns, preferably fully passively by the force of gravity and/or a wicking structure, into the thermal management fluid in contact with the heat-generating component. Thus, in a preferred feature of the invention, the step of transferring heat from the heat-generating component to the thermal management fluid of the present invention, including each of Compositions 1-38, causes the thermal management fluid to vaporize.

Examples of passive recirculating systems include a heat pipe or a thermosyphon. Such systems passively recirculate the thermal management fluid of the present invention, including each of Compositions 1-38, using gravity. In such a system, the thermal management fluid is heated by the heat-generating component, resulting in a heated thermal management fluid which is less dense and more buoyant. This thermal management fluid travels to a storage container, such as a tank where it cools and condenses. The cooled thermal management fluid then flows back to the heat source.

The electronic device includes a heat-generating component. The heat-generating component can be any component that includes an electronic element that generates heat as part of its operation. For the purposes of this invention, the heat generating component includes but is not limited to: semiconductor integrated circuits (ICs), electrochemical cells, power transistors, resistors, and electroluminescent elements, such as microprocessors, wafers used to manufacture semiconductor devices, power control semiconductors, electrical distribution switch gear, power transformers, circuit boards, multi-chip modules, packaged or unpackaged semiconductor devices, semiconductor integrated circuits, fuel cells, lasers (conventional or laser diodes), light emitting diodes (LEDs), and electrochemical cells, e.g. used for high power applications such as, for example, hybrid or electric vehicles.

For the purpose of this invention, the electronic device includes but is not limited to: personal computers, microprocessors, servers, cell phones, tablets, digital home appliances (e.g. televisions, media players, games consoles etc.), personal digital assistants, Datacenters, batteries both stationary and in vehicles, hybrid or electric vehicles, wind turbine, train engine, or generator. Preferably the electronic device is a hybrid or electric vehicle.

The present invention further relates to an electronic device comprising a thermal management fluid of the invention, including each of Compositions 1-38. For the purposes of this invention, the thermal management fluid is provided for cooling and/or heating the electronic device.

The present invention further relates to an electronic device comprising a heat generating component and a thermal management fluid of the invention, including each of Compositions 1-38. For the purposes of this invention, the electronic device can further comprise a heat exchanger, particularly where the heat exchanger is in contact with at least a part of the heat generating component.

The present invention further relates to an electronic device comprising a heat generating component, a heat exchanger, a pump and a thermal management fluid of the invention, including each of Compositions 1-38.

For the purposes of this invention, the heat generating component can be selected from semiconductor integrated circuits (ICs), electrochemical cells, power transistors, resistors, and electroluminescent elements, such as microprocessors, wafers used to manufacture semiconductor devices, power control semiconductors, electrical distribution switch gear, power transformers, circuit boards, multi-chip modules, packaged or unpackaged semiconductor devices, semiconductor integrated circuits, fuel cells, lasers (conventional or laser diodes), light emitting diodes (LEDs), and electrochemical cells, e.g. used for high power applications such as, for example, hybrid or electric vehicles.

For the purpose of this invention, the electronic device can be selected from personal computers, microprocessors, servers, cell phones, tablets, digital home appliances (e.g. televisions, media players, games consoles etc.), personal digital assistants, Datacenters, hybrid or electric vehicles, batteries both stationary and in vehicles, wind turbine, train engine, or generator, preferably wherein the electronic device is a hybrid or electric vehicle.

The invention further relates to the use of a thermal management fluid of the invention, including each of Compositions 1-38, for cooling an electronic device. For the purpose of this invention, the electronic device can be selected from personal computers, microprocessors, servers, cell phones, tablets, digital home appliances (e.g. televisions, media players, games consoles etc.), personal digital assistants, Datacenters, hybrid or electric vehicles, batteries both stationary and in vehicles, wind turbine, train engine, or generator, preferably wherein the electronic device is a hybrid or electric vehicle.

### Uses of Refrigerant and Heat Transfer Composition

The invention also provides a heat transfer system comprising a refrigerant or a heat transfer composition of the invention. It will be appreciated that the heat transfer systems described herein may be vapor compression systems having an evaporator, a condenser and a compressor in fluid communication.

The refrigerant or heat transfer composition of the invention, including each of Compositions 1-38, may be used as a secondary fluid.

It will be appreciated that the refrigerant or heat transfer composition of the invention, including each of Compositions 1-38 may be used in a variety of different heat transfer applications.

### Organic Rankine Cycle

As discussed above, when a heat transfer fluid of the present invention, including each of Compositions 1-38, is used in an Organic Rankine cycle, it is referred to as a working fluid. The working fluid therefore corresponds to the heat transfer fluid as discussed in this application. All preferred features of the heat transfer fluid apply to the working fluid as described herein.

Rankine cycle systems are known to be a simple and reliable means to convert heat energy into mechanical energy in the form of shaft power. In industrial settings, it may be possible to use flammable working fluids such as toluene and pentane, particularly when the industrial setting has large quantities of flammables already on site in processes or storage. However, for instances where the risk associated with use of a flammable and/or toxic working fluid is not acceptable, such as power generation in populous areas or near buildings, it is necessary or at least highly desirable to use non-flammable and/or non-toxic refrigerants as the working fluid. There is also a drive in the industry for these materials to be environmentally acceptable in terms of GWP.

The process for recovering waste heat in an Organic Rankine cycle according to the present invention preferably involves pumping liquid-phase working fluid of the present invention, including each of Compositions 1-38, through a boiler where an external (waste) heat source, such as a process stream, heats the working fluid causing it to evaporate into a saturated or superheated vapor. This vapor is expanded through a turbine wherein the waste heat energy is converted into mechanical energy. Subsequently, the vapor phase working fluid is condensed to a liquid and pumped back to the boiler in order to repeat the heat extraction cycle.

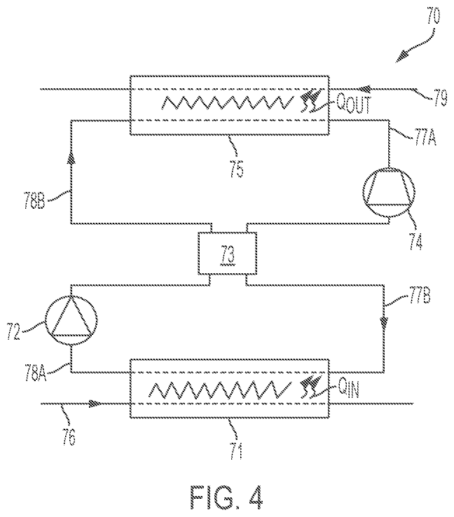

Referring to FIG. 4, in an exemplary organic Rankine cycle system 70, working fluid of the present invention, including each of Compositions 1-38, is circulated between an evaporator 71 and a condenser 75, with a pump 72 and an expansion device 74 functionally disposed therebetween. In the illustrated embodiment, an external flow of fluid is directed to evaporator 71 via external warm conduit 76. External warm conduit 76 may carry fluid from a warm heat source, such as a waste heat source from industrial processes (e.g., power generation), flue gases, exhaust gases, geothermal sources, etc.

Evaporator 71 is preferably configured as a heat exchanger which may include, e.g., a series of thermally connected, but fluidly isolated, tubes carrying fluid from warm conduit 76 and fluid from working fluid conduit 77B respectively. Thus, evaporator 71 facilitates the transfer of heat Q_{IN} from the warm fluid arriving from external warm conduit 76 to the relatively cooler (e.g., “cold”) working fluid arriving from expansion device 74 via working fluid conduit 77B.

The working fluid of the present invention, including each of Compositions 1-38, thus exits from evaporator 71, having been warmed by the absorption of heat Q_{IN}, and then travels through working fluid conduit 78A to pump 72. Pump 72 pressurizes the working fluid, thereby further warming the fluid through external energy inputs (e.g., electricity). The resulting “hot” fluid passes to an input of condenser 75 via conduit 78B, optionally via a regenerator 73 as described below.

Condenser 75 is configured as a heat exchanger similar to evaporator 71, and may include, e.g., a series of thermally connected, but fluidly isolated, tubes carrying fluid from cool conduit 79 and fluid from working fluid conduit 78B respectively. Condenser 75 facilitates the transfer of heat Q_{OUT} to the cool fluid arriving from external cool conduit 79 to the relatively warmer (e.g., “hot”) working fluid of the present invention, including each of Compositions 1-38, arriving from pump 72 via working fluid conduit 78B.

The working fluid of the present invention, including each of Compositions 1-38, exiting from condenser 75, having thus been cooled by the loss of heat _{OUT}, then travels through working fluid conduit 77A to expansion device 74. Expansion device 74 allows the working fluid to expand, thereby further cooling the fluid. At this stage, the fluid of the present invention, including each of Compositions 1-38, may perform work, e.g., by driving a turbine. The resulting “cold” fluid passes to an input of evaporator 71 via conduit 77B, optionally via a regenerator 73 as described below, and the cycle begins anew.

Thus, working fluid conduits 77A, 77B, 78A and 78B define a closed loop such that the working fluid contained therein may be reused indefinitely, or until routing maintenance is required.

In the illustrated embodiment, regenerator 73 may be functionally disposed between evaporator 71 and condenser 75. Regenerator 73 allows the “hot” working fluid of the present invention, including each of Compositions 1-38, exiting from pump 72 and the “cold” working fluid issued from expansion device 74 to exchange some heat, potentially with a time lag between deposit of heat from the hot working fluid and release of that heat to the cold working fluid. In some applications, this can increase the overall thermal efficiency of Rankine cycle system 70.

Therefore, the invention relates to an organic Rankine cycle comprising a working fluid of the present invention, including each of Compositions 1-38.

The invention further relates to the use of a working fluid of the invention, including each of Compositions 1-38, in an Organic Rankine Cycle.

The invention also provides a process for converting thermal energy to mechanical energy in a Rankine cycle, the method comprising the steps of i) vaporizing a working fluid of the invention, including each of Compositions 1-38, with a heat source and expanding the resulting vapor, then ii) cooling the working fluid with a heat sink to condense the vapor, wherein the working fluid is a refrigerant or heat transfer composition of the invention, including each of Compositions 1-38.

The mechanical work may be transmitted to an electrical device such as a generator to produce electrical power.

The heat source maybe provided by, for example, a thermal energy source selected from industrial waste heat, solar energy, geothermal hot water, low pressure steam, distributed power generation equipment utilizing fuel cells, prime movers, or an internal combustion engine. The low pressure steam is preferably a low pressure geothermal steam or is provided by a fossil fuel powered electrical generating power plant.

The heat source is preferably provided by a thermal energy source selected from industrial waste heat, or an internal combustion engine.

It will be appreciated that the heat source temperatures can vary widely, for example from about 90° C. to >800° C., and can be dependent upon a myriad of factors including geography, time of year, etc. for certain combustion gases and some fuel cells.

Systems based on sources such as waste water or low pressure steam from, e.g., a plastics manufacturing plants and/or from chemical or other industrial plant, petroleum refinery, and related word forms, as well as geothermal sources, may have source temperatures that are at or below about 175° C. or at or below about 100° C., and in some cases as low as about 90° C. or even as low as about 80° C. Gaseous sources of heat such as exhaust gas from combustion process or from any heat source where subsequent treatments to remove particulates and/or corrosive species result in low temperatures may also have source temperatures that are at or below 200° C., at or below about 175° C., at or below about 130° C., at or below about 120° C., at or below about 100° C., at or below about 100° C., and in some cases as low as about 90° C. or even as low as about 80° C.

However, it is preferred in some applications that the heat source has a temperature of at least about 200° C., for example of from about 200° C. to about 400° C.

In an alternative preferred embodiment, the heat source has a temperature of from 400 to 800° C., more preferably 400 to 600° C.

### Heat Pump

As discussed above, when a heat transfer fluid of the present invention, including each of Compositions 1-38, used in a heat pump, it is referred to as a refrigerant. The refrigerant therefore corresponds to the heat transfer fluid as discussed this application. All preferred features of the heat transfer fluid as described apply to the refrigerant as described herein.

The refrigerant or heat transfer composition of the invention, including each of Compositions 1-38, may be used in a high temperature heat pump system.

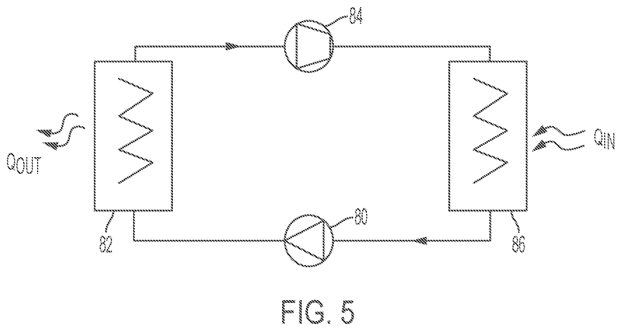

Referring to FIG. 5, in one exemplary heat pump system, compressor 80, such as a rotary, piston, screw, or scroll compressor, compresses a refrigerant of the present invention, including each of Compositions 1-38, which is conveyed to a condenser 82 to release heat Q_{OUT} to a first location, followed by passing the refrigerant through an expansion device 84 to lower the refrigerant pressure, followed by passing the refrigerant through an evaporator 86 to absorb heat Q_{IN} from a second location. The refrigerant is then conveyed back to the compressor 80 for compression.

The present invention provides a method of heating a fluid or body using a high temperature heat pump, said method comprising the steps of (a) condensing a refrigerant composition of the present invention, including each of Compositions 1-38, in the vicinity of the fluid of body or be heated, and (b) evaporating said refrigerant.

Examples of high temperature heat pumps include a heat pump tumble dryer or an industrial heat pump. It will be appreciated the heat pump may comprise a suction line/liquid line heat exchanger (SL-LL HX). By “high temperature heat pump”, it is meant a heat pump that is able to generate temperatures of at least about 80° C., preferably at least about 90° C., preferably at least about 100° C., more preferably at least about 110° C.

### Secondary Loop System

As discussed above, when the heat transfer fluid of the present invention, including each of Compositions 1-38, is used in a secondary loop system, it is referred to as a refrigerant.

The refrigerant of the present invention, including each of Compositions 1-38, may be used as secondary refrigerant fluid in a secondary loop system.

A secondary loop system contains a primary vapor compression system loop that uses a primary refrigerant and whose evaporator cools the secondary loop fluid. The secondary refrigerant fluid, including each of Compositions 1-38, then provides the necessary cooling for an application. The secondary refrigerant fluid should preferably be non-flammable and have low-toxicity since the fluid in such a loop is potentially exposed to humans in the vicinity of the cooled space. In other words, the refrigerant or heat transfer composition of the present invention, including each of Compositions 1-38, may be used as a “secondary refrigerant fluid” in a secondary loop system.

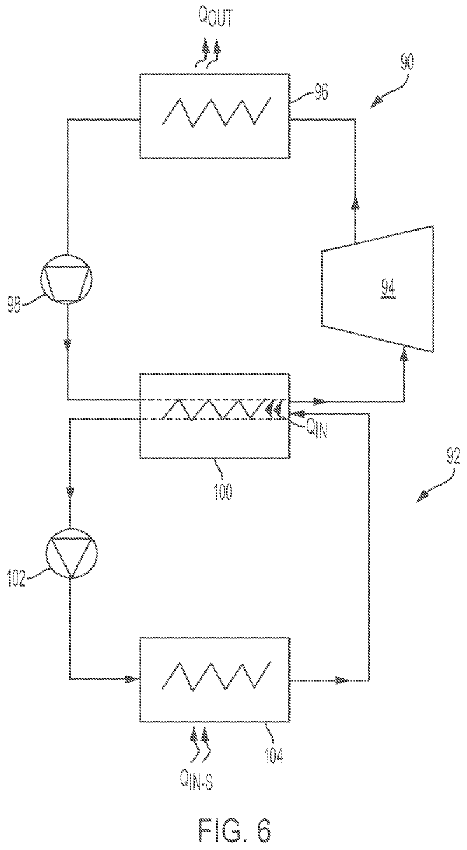

Referring to FIG. 6, one exemplary secondary loop system includes a primary loop 90 and a secondary loop 92. In primary loop 90, compressor 94, such as a rotary, piston, screw, or scroll compressor, compresses a primary refrigerant, which is conveyed to a condenser 96 to release heat Q_{OUT} to a first location, followed by passing the primary refrigerant through an expansion device 98 to lower the refrigerant pressure, followed by passing the primary refrigerant through a refrigerant/secondary fluid heat exchanger 100 to exchange heat Q_{IN} with a secondary fluid, including each of Compositions 1-38, with the secondary fluid pumped through secondary loop 92 via a pump 102 to a secondary loop heat exchanger 104 to exchange heat with a further location, for example to absorb heat Q_{IN-S} to providing cooling to the further location.

The primary fluid used in the primary loop (vapor compression cycle, external/outdoors part of the loop) may be selected from but not limited to HFO-1234ze(E), HFO-1234yf, propane, R455A, R32, R466A, R44B, R290, R717, R452B, R448A, and R449A, preferably HFO-1234ze(E), HFO-1234yf, or propane.

The secondary loop system may be used in refrigeration or air conditioning applications, that is, the secondary loop system may be a secondary loop refrigeration system or a secondary loop air conditioning system.

Examples of refrigeration systems which can include a secondary loop refrigeration systems that include a secondary refrigerant of the present invention, including each of Compositions 1-38, include:
a low temperature refrigeration system,
a medium temperature refrigeration system,
a commercial refrigerator,
a commercial freezer,
an industrial freezer,
an industrial refrigerator and
a chiller.

Examples of air conditioning systems which can include a secondary loop air conditioning system which utilize a refrigerant of the present invention, including each of Compositions 1-38, include in mobile air conditioning systems or stationary air conditioning systems. Mobile air-conditioning systems including air conditioning of road vehicles such as automobiles, trucks and buses, as well as air conditioning of boats, and trains. For example, where a vehicle contains a battery or electric power source.

Examples of stationary air conditioning systems which can include a secondary loop air conditioning system which utilize a refrigerant of the present invention, including each of Compositions 1-38, include:
a chiller, particularly a positive displacement chiller, more particularly an air cooled or water-cooled direct expansion chiller, which is either modular or conventionally singularly packaged,
a residential air conditioning system, particularly a ducted split or a ductless split air conditioning system,
a residential heat pump,
a residential air to water heat pump/hydronic system,
an industrial air conditioning system
a commercial air conditioning system, particularly a packaged rooftop unit and a variable refrigerant flow (VRF) system;
a commercial air source, water source or ground source heat pump system.

A particularly preferred heat transfer system according to the present invention is an automotive air conditioning system comprising a vapour compression system (the primary loop) and a secondary loop air conditioning system, wherein the primary loop contains HFO-1234yf as the refrigerant and the second loop contains a refrigerant or heat transfer composition of the present invention, including each of Compositions 1-38. In particular, the secondary loop can be used to cool a component in the car engine, such as the battery.

It will be appreciated the secondary loop air conditioning or refrigeration system may comprise a suction line/liquid line heat exchanger (SL-LL HX).

### Methods

The present heat transfer fluids, or heat transfer compositions which can include a secondary loop air conditioning system which utilize a refrigerant of the present invention, including each of Compositions 1-38, may be used as a replacement for existing fluids.

The invention includes a method of replacing an existing heat transfer fluid in a heat transfer system, said method comprising the steps of (a) removing at least a portion of said existing heat transfer fluid from said system, and subsequently (b) introducing into said system a heat transfer fluid of the invention. Step (a) may involve removing at least about 5 wt. %, at least about 10 wt. %, at least about 15 wt. %, at least about 50 wt. % at least about 70 wt. %, at least about 90 wt. %, at least about 95 wt. %, at least about 99 wt. % or at least about 99.5 wt. % or substantially all of said existing heat transfer fluid from said system prior to step (b).

The method may optionally comprise the step of flushing said system with a solvent after conducting step (a) and prior to conducting step (b).

For the purposes of this invention, the heat transfer fluid of the present invention, including each of Compositions 1-38, can be used to replace an existing fluid in an electronic device, in an Organic Rankine cycle, in a high temperature heat pump or in a secondary loop.

For example, the thermal management fluid of the invention, including each of Compositions 1-38, may be used as a replacement for existing fluids such as HFC-4310mee, HFE-7100 and HFE-7200. Alternatively, the thermal management fluid, including each of Compositions 1-38, can be used to replace water and glycol. The replacement may be in existing systems, or in new systems which are designed to work with an existing fluid. Alternatively, the thermal management fluid, including each of Compositions 1-38, can be used in applications in which the existing refrigerant was previously used. For example, the refrigerants of the invention, including each of Compositions 1-38, may be used as a replacement for existing refrigerants such as HFC-245fa, HFC-134a, HFC-404A and HFC-410A. The refrigerant, including each of Compositions 1-38, may be used in applications in which the existing refrigerant was previously used. Alternatively, the refrigerant of the present invention, including each of Compositions 1-38, may be used to retrofit an existing refrigerant in an existing system. Alternatively, the refrigerant of the present invention, including each of Compositions 1-38, may be used in new systems which are designed to work with an existing refrigerant.

The invention provides a method of replacing an existing refrigerant in a heat transfer system, said method comprising the steps of (a) removing at least a portion of said existing refrigerant from said system, and subsequently (b) introducing into said system a refrigerant of the invention of the present invention, including each of Compositions 1-38. The existing refrigerants may be selected, for example, from HFC-245fa, HFC-134a, HFC-404A and HFC-410A.

Step (a) may involve removing at least about 5 wt. %, at least about 10 wt. %, at least about 15 wt. %, at least about 50 wt. % at least about 70 wt. %, at least about 90 wt. %, at least about 95 wt. %, at least about 99 wt. % or at least about 99.5 wt. % of said existing refrigerant from said system prior to step (b).

The method may optionally comprise the step of flushing said system with a solvent after conducting step (a) and prior to conducting step (b).

### EXAMPLES

### Example 1—Organic Rankine Cycle

This example illustrates that the compositions of the present invention, including each of Compositions 1-38, are useful as a working fluid in an Organic Rankine cycle based on a comparison of the estimated thermal efficiency of various working fluids in an organic Rankine cycle. In this example, an ORC system is assumed to contain a condenser, pump, boiler and turbine and the following qualitative results will occur as shown in Table 1 below.

TABLE 1

| | | Process Specifications | | | | | | |
|---|---|---|---|---|---|---|---|---|
| | | | | Boiler | | Condens- | | |
| | | Boiler | Critical | Super- | Isentropic | ing | Isentropic | Estimated |
| | Working | Temp | Temp | heat | Efficiency | Temp | Efficiency | Thermal |
| | Fluid | (° C.) | (° C.) | (° C.) | (Turbine) | (° C.) | (Pump) | Efficiency |
| Ex. | Compositions | 144 | 225 | 1 | 0.8 | 35 | 0.8 | 15-20% |
| 1A | 1-38 | | | | | | | |
| | R245fa | 144 | 154 | 1 | 0.8 | 35 | 0.8 | 15.41% |
| | R1233zd(E) | 144 | 166 | 1 | 0.8 | 35 | 0.8 | 15.92% |
| | TFMCB | | | | | | | |
| Ex. | Compositions | 210 | 225 | 1 | 0.8 | 35 | 0.8 | 15-20% |
| 1B | 1-38 | | | | | | | |
| | R245fa | 144 | 154 | 1 | 0.8 | 35 | 0.8 | 15.41% |
| | R1233zd(E) | 156 | 166 | 1 | 0.8 | 35 | 0.8 | 16.66% |

### Example 2—Compositions 1-38 Compared to Novec 7200 in a Heat Exchanger

Batteries of electric vehicles develop heat during operation when charging and discharging. The typical design of vehicle batteries differs between three types: cylindrical cells, pouch cells and prismatic cells. All three types have different considerations in terms of heat transfer due to their shape. Prismatic and pouch cells are often used with cooling plates due to the straight outer faces. Cylindrical cells employ cooling ribbons that are in thermal contact with the outer shell of the cells. Extensive heat generation during charging and discharging of the cells can lead to an increase in temperature that can cause decreasing performance and reduced battery lifetime.

A battery cooling plate set up may be used to provide active cooling to a battery and remove the heat (e.g. to remove heat from the battery of an electric vehicle). In this Example, the performance of fluids of the present invention, including each of Compositions 1-38 and 3M Novec 7200 is analyzed for their ability to provide cooling in single phase heat transfer.

It will be appreciated that the convective heat transfer can occur either by direct contact, I.e. when the battery is immersed in the fluid that may be pumped through the battery enclosure or indirectly, i.e. by using a cooling plate with a combination of convective and conductive heat transfer.

The present example uses a round tube with an internal diameter of 0.55 inches to provide a cooling load of 10246 BTU/h (3 kW). The tube length was 30 ft (9.14 m) with an assumed pressure drop of 2.9 PSI (20 kPa). The fluid temperature was 7.2 C (45 F). The internal heat transfer coefficient is determined for turbulent flow. The necessary mass flow rate to remove the cooling load is determined for both fluids. The results of the comparison are shown in the Table below. It can be seen in the results that the necessary mass flow rate to remove the generated heat is about or less than for 3M Novec 7200 and that the useful output (I.e. the heat transfer coefficient) is about or higher than 3M Novec 7200.

TABLE
Heat Transfer and Pressure Drop For Heat Exchanger Set Up

| | Mass Flow | Prandtl | Internal heat |
|---|---|---|---|
| | Rate | Number | transfer coefficient |
| | lb/s | [-] | BTU/(h-ft²-F) |
| Compositions | 0.9-1 | 9-11 | 300-350 |
| 1-38 | | | |
| 3M Novec 7200 | 0.98 | 10.4 | 303.4 |

### Example 3—Secondary AC System

The efficiency of secondary loop air conditioning system, as determined by the estimated coefficient of performance (COP), is evaluated for the use of heat each of Compositions 1-38 as a secondary refrigerant with R1234ze(E), R1234yf, and propane as primary refrigerant options. The system is composed of a vapor-compression primary loop and a pumped two-phase secondary loop that are thermally connected by an internal heat exchanger. This internal heat exchanger acted as an evaporator for the primary loop and a condenser for the secondary loop. Using the thermodynamic properties of the primary and secondary refrigerants at the specified conditions of each unit operation, defined in Table 2, the COP is evaluated relative to the performance of R410A in an air conditioning system.

TABLE 2
Operating Conditions

| Operating | T_{cond.} | T_{cond sink} | [symbol] T_{SC} | T_{evap.} | [symbol] evap sink | □T_{SH} | [symbol] Isentropic | [symbol] Volumetric | T_{IHX-SH} | [symbol] _{IHX-Sat} |
|---|---|---|---|---|---|---|---|---|---|---|
| Conditions | (° C.) | (° C.) | (° C.) | (° C.) | (° C.) | (° C.) | (—) | (—) | [symbol] ° C.) | [symbol] ° C.) |
| Basic Cycle (R410A) | 45 | 35 | −5 | 7 | 27 | +5 | 70% | 100% | N/A | N/A |
| Secondary Cycle | 45 | 35 | −5 | 7 | 27 | (flooded) | 70% | 100% | +5 | +5 |
| [symbol] ″/ | | | | | | | | | | |
| Composition 1-38 | | | | | | | | | | |

Nomenclature: T = Temperature, □ = Efficiency, □ = Difference, SC = Sub-cooling, SH = Superheat, IHX = Intermediate Heat Exchanger, Sat = Saturation [symbol]
indicates data missing or illegible when filed

TABLE 3
Performance of secondary AC cycle

| Primary | | | | | |
|---|---|---|---|---|---|
| Refrigerant | Secondary | GWP | GWP | | |
| (“X”) | Refrigerant | Primary | Secondary | Capacity | Efficiency |
| R410A | | 1924 | | 100% | 100% |
| R1234ze(E) | Compositions | <1 | <1-150 | 100% | 90-95% |
| | 1-38 | | | | |
| R1234yf | Compositions | <1 | <1-150 | 100% | 90-95% |
| | 1-38 | | | | |
| Propane | Compositions | 3 | <1-150 | 100% | 90-95% |
| | 1-38 | | | | |

Table 3 shows the thermodynamic performance of the secondary AC system with different primary refrigerants and using each of Compositions 1-38 as secondary refrigerant, with the capacity of the secondary AC system being matched to R410A system in all the cases.

### Example 4—High Temperature Heat Pump Application Using Compositions 1-38

High temperature heat pumps can utilize waste heat and provide high heat sink temperatures. Compositions 1-38 of the present invention each provide efficiency equal to about or superior to R245fa over a wide range of condensing temperatures.

### Operating Conditions:

Condensing temperature varied between 90° C., 100° C. and 110° C.
Subcooling: 10° C.
Evaporating temperature: 25° C.
Evaporator Superheat: 15° C.
Isentropic efficiency: 65%

TABLE 4
Relative heating COP at varying condensing temperatures

| Fluid | Condensing temperature | | |
|---|---|---|---|
| Fluid | 90° C. | 100° C. | 110° C. |
| R245fa | 100.0% | 100.0% | 100.0% |
| Compositions | 100-105% | 100-105% | 100-105% |
| 1-38 | | | |

### Example 5—Thermodynamic Performance of a Secondary Loop Medium Temperature Refrigeration System

The efficiency of secondary loop medium temperature refrigeration system, as determined by the estimated coefficient of performance (COP), is evaluated for the use of each of Compositions 1-38 as a secondary refrigerant with R1234ze(E), R1234yf, and propane as primary refrigerant options. The system is composed of a vapor-compression primary loop and a pumped two-phase secondary loop that are thermally connected by an internal heat exchanger. This internal heat exchanger acts as an evaporator for the primary loop and a condenser for the secondary loop. The COP was evaluated relative to the performance of R134a in an air conditioning system and the each of Compositions 1-38 about matches or is superior to the efficiency of R134a.

### Example 6—Sensible Heat Immersion Cooling Application Using Compositions 1-38

Batteries of electric vehicles develop heat during operation when charging and discharging. The typical design of vehicle batteries differs between three types: Cylindrical cells, pouch cells and prismatic cells. All three types have different considerations in terms of heat transfer due to their shape. Extensive heat generation during charging and discharging of the cells can lead to an increase in temperature that can cause decreasing performance and reduced battery lifetime.

Compositions 1-38 of the present invention have low dielectric constants, high dielectric strength, and are nonflammable fluids, which allows for direct cooling of the battery cells that are immersed in each of Compositions 1-38.

The present example considers a battery module that consists of 1792 cylindrical battery cells of 18650 type. In one case the battery module is cooled by a 50/50 mixture of water/glycol in a flat tube heat exchanger that is on contact with the battery cells. In the other case the cells are immersed in each of Compositions 1-38, i.e. are in direct contact with the fluid. The waste heat for the battery module is 8750 W that is evenly distributed over the total number of cells. The assumptions and operating conditions are listed in Table 5.

TABLE 5
Assumptions for Battery module design and operating conditions

| | | Water/ | Compositions |
|---|---|---|---|
| Parameter | Unit | Glycol | 1-38 |
| Battery diameter | [mm] | 18.5 | 18.5 |
| Battery gap | [mm] | 3.8 | 1.5 |
| Battery height | [mm] | 65 | |
| Number of batteries | [-] | 1792 | |
| Battery mass | [g] | 49 | |
| Battery specific heat | [J/ | 830 | |
| | kgK] | | |
| Total battery module waste heat | [W] | 8750 | |
| Fluid flow rate | [kg/s] | 0.1 | |
| Initial module temperature | [° C.] | 30 | |
| Fluid inlet temperature | [° C.] | 10 | |
| Cooling channel height | [mm] | 30 | n/a |
| Cooling channel width | [mm] | 2.8 | n/a |
| Heat exchanger flat tube wall thickness | [mm] | 0.5 | n/a |
| Heat exchanger flat tube | [W/ | 3 | n/a |
| thermal conductivity | mK] | | |
| Heat exchanger flat tube relative | [-] | 0.0003 | n/a |
| surface roughness | | | |

TABLE 6
Minimum and maximum cell temperatures in battery module

| | Minimum cell temperature [° C.] | | Maximum cell temperatures [° C.] | |
|---|---|---|---|---|
| | Water/Glycol | Compositions | Water/Glycol | Compositions |
| Time | 50/50 | 1-38 | 50/50 | 1-38 |
| 0 | 30.0 | 30.0 | 30.0 | 30.0 |
| 100 | 35.8 | 10-40 | 36.8 | 30-40 |
| 200 | 40.3 | 10-40 | 42.0 | 30-40 |
| 300 | 43.6 | 10-45 | 46.0 | 30-45 |
| 400 | 46.1 | 10-45 | 49.2 | 30-50 |
| 500 | 48.0 | 10-45 | 51.7 | 30-50 |
| 600 | 49.5 | 10-50 | 53.6 | 30-55 |
| 700 | 50.5 | 10-50 | 55.1 | 30-55 |
| 800 | 51.4 | 10-50 | 56.3 | 30-55 |
| 900 | 52.0 | 10-50 | 57.2 | 30-55 |

### Example 10—Two Phase Immersion Cooling Application Using Compositions 1-38 in a Data Center

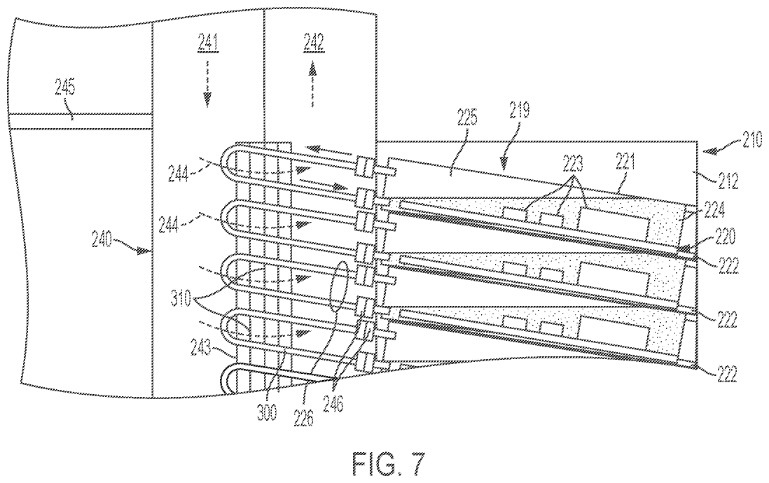

An example of data center cooling is provided, making reference to FIG. 7. A data center, generally denoted 200, includes a plurality of electronic subsystems 220 contained in one or more of electronics racks 210. At least one, and preferably a plurality, and preferably all, of the electronic subsystems 220 are associated with a cooling station 240 that includes (in one embodiment) a vertically-extending, liquid-to-air heat exchanger 243 and supply and return ducting 241, 242 for directing a cooling airflow 244 across liquid-to-air heat exchanger 243. A cooling subsystem 219 is associated with at least one, and preferably a plurality, and preferably all, of the multiple electronic subsystems 220. In a preferred embodiment, as shown in FIG. 7, all of the subsystem 220 are associated with the cooling station 240 and a cooling subsystem 219. Each cooling subsystem 219 comprises (in this embodiment) a housing 221 (which preferably is a low pressure housing) which encloses a respective electronic subsystem 220 comprising a plurality of electronic components 223. The electronic components are in operation as part of the data center and are generating heat as a result of performing their function in the data center. The components include, by way of example, printed circuit boards, microprocessor modules, and memory devices. Each electronic subsystem has, as it is operating, its heat generating components immersed in a thermal management fluid of the present invention 224, including each of Compositions 1-38. The fluid 224 boils in typical operation, generating dielectric vapor 225 according to the present invention. In the illustrated embodiment, electronic subsystems 220 are angled by providing upward-sloped support rails 222 within electronics rack 210 to accommodate the electronic subsystems 220 at an angle. Angling of the electronic subsystems as illustrated facilitates buoyancy-driven circulation of vapor 225 between the cooling subsystem 219 and the liquid-to-air heat exchanger 243 of the associated local cooling station 240. However, the excellent results according to the present invention and the present example are achieved equally well when such angling is not used. Multiple coolant loops 226 are coupled in fluid and thermal contact with the liquid-cooled electronic subsystems and a respective portion of liquid-to-air heat exchanger 243. In particular, multiple tubing sections 300 pass through liquid-to-air heat exchanger 243, which in this example includes a plurality of air-cooling fins 310. Vapor 225 is buoyancy-driven from housing 221 to the corresponding tubing section 300 of liquid-to-air heat exchanger 243, where the vapor condenses and is then returned as liquid to the associated liquid-cooled electronics subsystem. Cooling airflow 244 is provided in parallel to the supply ducting 241 of multiple local cooling stations 240 of data center 200, and the heated airflow is exhausted via return ducting 242. The equipment as described herein, but not the fluid of the present invention, is disclosed in US 2013/0019614, which is incorporated herein by reference.

The system as describe above is operated with a thermal management fluid consisting of the present invention, including each of Compositions 1-38, and ambient air as the heat sink for the condenser, and this system operates to effectively, efficiently, safely and reliably maintain the electronic components in the most desired operating temperature range while the system is performing its function in the operating data center.

### Example 11—Preparation of 1-chloro-2,3,3-tetrafluorocyclobut-1-ene (Compound 1)

In a three-necked 1 liter flask equipped with a mechanical stirrer, a vigreux column, and an addition funnel, was charged with 74 g zinc powder, 220 ml 1,4-dioxane, and 0.5 g ZnCl₂. The mixture was heated to 95° C., and 212.7 g of 1,1,2-trichloro-2,3,3-trifluorocyclobutane was added dropwise with stirring via the addition funnel. The product was distilled out via the vigreux column then redistilled through a packed column to give 121.4 g 99% 1-chloro-2,3,3-tetrafluorocyclobut-1-ene (Compound 1) in a 85.4% yield.

### Example 12—Preparation of 1,3,4-trifluoro-2,3-bis(trifluoromethyl)cyclobut-1-ene (Compound 8)

The contents of a three-necked 1-L flask equipped with a mechanic stirrer, vigreux column, and an addition funnel, charged with 100 g KOH powder, 100 g CaO powder, 1.2 g t-butyl catechol, 1.2 g diphenylamine, and 2.5 g aliquat 336 was heated to 90-100° C. with stirring. 110 g of 1,2,3,4-tetrafluoro-1,2-bis(trifluoromethyl)cyclobutane was then added dropwise via the addition funnel and the following reaction occurred:

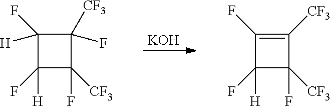

The reaction product is continuously distilled out and collected in a dry-ice trap, which contained 0.2 g of t-butyl catechol. A total of 9 liters of clear liquid is collected after 3 hours at 100° C. GC analysis is performed and shows 65% cis and trans mixture of 1,3,4-trifluoro-2,3-bis(trifluoromethyl)cyclobut-1-ene (Compound 8A and 8B respectfully), 35% of the starting material together with other by-products.

### Example 13—Preparation of 1,3,3,4,4-pentafluoro-2-((1,1,1,3,3,3-hexafluoropropan-2-yl)oxy)cyclobut-1-ene (Compound 14 and 1,3,3,4,4-pentafluoro-1,2-bis((1,1,1,3,3,3-hexafluoropropan-2-yl)oxy)cyclobut-1-ene (Compound 15)

A solution of 85% potassium hydroxide (35 g, 0.53 mole based on 85% purity) was prepared in 200 g. of 1,1,1,3,3,3-hexafluoropropanol and charged to a 600 ml stainless steel autoclave which was then sealed, cooled with dry-ice and evacuated. 81.6 g of perfluorocyclobutene (0.5 mol) was vacuum transferred into the autoclave. After warming to room temperature, the autoclave was slowly heated to 60° C. and stirred overnight. A pressure of 30 psig was developed and then rapidly decreased. After 18 hours the autoclave was opened in the hood and the contents were poured into 1000 ml water in a separator funnel, the lower organic layer was separated, dried, and distilled. After distillation, the recovered products were: 20 g of starting material; 75.6 g of 1,3,3,4,4-pentafluoro-2-((1,1,1,3,3,3-hexafluoropropan-2-yl)oxy)cyclobut-1-ene (Compound 14) at a yield of 45% (measured boiling of about 81-82° C.; and 110.8 g 99.4% pure 3,3,4,4-pentafluoro-1,2-bis((1,1,1,3,3,3-hexafluoropropan-2-yl)oxy)cyclobut-1-ene (Compound 15) in a 48% yield with a measured boiling of about 26-127° C. The structure of each product formed was confirmed by GCMS and NMR analysis.

### Example 14—Preparation of perfluorocyclobut-1-en-1-yl)oxy)benzene (Compound 16)

A solution of 85% potassium hydroxide (65 g, 1.0 mole based on 85% purity) was prepared in 200 ml. of phenol and charged to a 600 ml stainless steel autoclave which was then sealed, cooled and evacuated. 162 g of perfluorocyclobutene (1.0 mol) was vacuum transferred into the autoclave, and after warming, the autoclave was stirred at room temperature overnight. A pressure of 30 psig. developed and then rapidly decreased. After 18hours the autoclave was opened in the hood and the contents were poured into a separator funnel and washed with 250 ml water twice. The lower organic layer is separated, dried, and distilled. 200 g of ((perfluorocyclobut-1-en-1-yl) oxy) benzene (Compound 16) is obtained, boiling at 38-40° C./0.5torr. The structure of the recovered product is examined by GCMS and NMR analysis and confirmed to be Compound 16.

### Example 15—Preparation of ((perfluorocyclobut-1-ene-1,2-diyl)bis(oxy))dibenzene (Compound 17)

A solution of 85% potassium hydroxide (120 g, 1.82 mole based on 85% purity) was prepared in 300 ml. of phenol and charged to a 600 ml stainless steel autoclave which was then sealed, cooled and evacuated. 147 g of perfluorocyclobutene (0.91 mol) was vacuum transferred into the autoclave, and after stirring overnight at room temperature, autoclave was opened, and the reaction mixture was quenched into 1 liter of cold water. The organic layer was separated and the aqueous phase was extracted with methylene dichloride twice (2×200 ml). The combined organics were washed with saturated sodium chloride, dried (MgSO4), and distilled to give 242 g of ((perfluorocyclobut-1-ene-1,2-diyl)bis(oxy))dibenzene (Compound 17) at a yield of 86% and having a boiling point of 68-70° C. at 0.5 torr.

### Example 16—Preparation of 1-chloro-3,3,4,4-tetrafluoro-2-(2,2,2-trifluoroethoxy)cyclobut-1-ene (Compound 18)

A solution of 101.0 g (0.51 mol) of 1,2-dichlorotetrafluorocyclobutene in 100 g of 2,2,2-trifluoroethanol at room temperature was treated dropwise with a solution of 37.0 g (0.56 mol) of potassium hydroxide (85%) in 200 g of 2,2,2-trifluoroethanol. After the solution had been stirred for 24 h, the mixture was quenched in 1000 ml of dionized water, the resulting organic layer was dried and distilled to give 58.2 g 1-chloro-3,3,4,4-tetrafluoro-2-(2,2,2-trifluoroethoxy)cyclobut-1-ene (Compound 18).

1-18. (canceled)

19. A heat transfer composition for transferring heat and/or energy to and/or from an article, device or fluid, wherein said heat transfer fluid comprises, consists essentially of or consists of a compound according to Formula I or Formula II or Formula 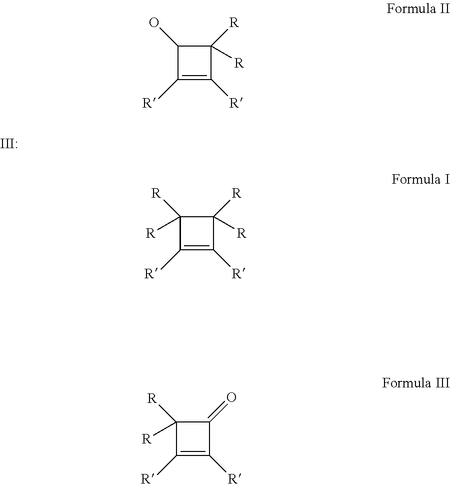
where each R and each R′ is independently selected from H, F, Cl, R1, and OR1, each R1 is independently: a C1 to C3 alkane that is unsubstituted or substituted with F and/or Cl; or C1 to C3 alkene that is unsubstituted or substituted with F and/or Cl; or Bz, where Bz is an unsubstituted benzene ring, and provided that the molecule has at least three F substituents bonded to a carbon in the cyclobutene ring.

20. The heat transfer composition of claim 19 wherein said compound comprises a compound according to Formula I, provided that no R′ is Cl.

21. The heat transfer composition according to claim 19 having a boiling point of from about 25° C. to about 150° C. and a dielectric constant of less than about 8.

22. The compound ((perfluorocyclobut-1-ene-1,2-diyl)bis(oxy))dibenzene and having the following structure:

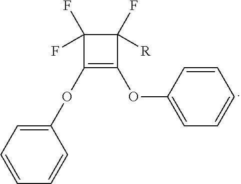

23. A heat transfer composition comprising a compound according to claim 22.

24. A cooled electronic component in heat transfer contact with a composition according to claim 19.

25. A vapour compression heat transfer system comprising a composition according to claim 19.

26. An air conditioning system comprising a composition according to claim 19.

27. A battery and/or a battery cooling system comprising a composition according to claim 19.

28. The battery and/or a battery cooling system according to claim 27 located in and providing motive force for a vehicle.
